(19) United States
(12) Patent Application Publication
(10) Pub. No.: US 2025/0205483 A1
(43) Pub. Date: Jun. 26, 2025

(54) DETERMINING TRANSDUCER LOCATIONS FOR DELIVERY OF TUMOR TREATING FIELDS USING EXTERNAL MEASUREMENTS OF SUBJECT AND USING SUBJECT SELECTION TREE

(71) Applicant: Novocure GmbH, Root D4 (CH)

(72) Inventors: Oren PELES ZEEVI, Haifa (IL); Amit FELDMAN, Haifa (IL)

(73) Assignee: Novocure GmbH, Root D4 (CH)

(21) Appl. No.: 18/986,839

(22) Filed: Dec. 19, 2024

Related U.S. Application Data
(63) Continuation-in-part of application No. 18/960,626, filed on Nov. 26, 2024.
Application No. 18/960,626 is a continuation-in-part of application No. 18/961,009, filed on Nov. 26, 2024.
(60) Provisional application No. 63/567,837, filed on Mar. 20, 2024.
(60) Provisional application No. 63/613,858, filed on Dec. 22, 2023.
(60) Provisional application No. 63/613,835, filed on Dec. 22, 2023.

(51) Int. Cl. A61N 1/36; A61N 1/08

(52) U.S. Cl. CPC A61N 1/36002; A61N 1/08

(57) ABSTRACT

A method for determining transducer locations for delivering tumor treating fields based on models of healthy subjects, comprising: receiving a plurality of external measurements of the subject, the plurality of external measurements not derived from a medical image of the subject; receiving a selection of a general location of an abnormality of the subject, the general location selected from a plurality of general locations of the subject; selecting a healthy model from a plurality of healthy models, the selected healthy model being representative of the subject, the selection based on the external measurements of the subject; selecting locations on the selected healthy model to place transducers to treat the abnormality of the subject using tumor treating fields, the selection based on the selected general location of the abnormality of the subject; and providing the selected locations to place transducers to treat the abnormality of the subject using tumor treating fields.

### CROSS-REFERENCE TO RELATED APPLICATIONS

This application claims priority to U.S. patent application Ser. No. 18/960,626, filed Nov. 26, 2024, U.S. patent application Ser. No. 18/961,009, filed Nov. 26, 2024, U.S. Provisional Application No. 63/567,837, filed Mar. 20, 2024, U.S. Provisional Application No. 63/613,858, filed Dec. 22, 2023, and U.S. Provisional Application No. 63/613,835, filed Dec. 22, 2023, which are incorporated herein by reference in their entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into a region of interest by transducers placed on the patient's body and applying alternating current (AC) voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIG. 1A depicts an example method for determining transducer locations for delivery of tumor treating fields using simulations based on models of healthy subjects according to an embodiment.

FIG. 1B depicts an example method for generating healthy models according to an embodiment.

FIG. 2 depicts examples of healthy models.

FIG. 3 depicts an example plot used for clustering healthy models.

FIG. 4 depicts an example plot used for clustering healthy models.

FIG. 5 depicts an example plot of clusters of healthy models.

FIG. 6 depicts an example method for determining transducers locations based on external measurements of a subject according to an embodiment.

FIG. 7 depicts an example method of generating a plurality of healthy models according to an embodiment.

FIG. 8 depicts an example method of predetermining locations on healthy models to place transducers to deliver TTFields to a plurality of general locations for each healthy model according to an embodiment.

FIGS. 9A-9J depict examples of user interfaces for determining transducer locations for delivery of tumor treating fields using simulations based on models of healthy subjects according to an embodiment.

FIG. 10 depicts a method for determining transducer locations for delivery of tumor treating fields using category information and a subject selection tree according to an embodiment.

FIGS. 11A-11F depict examples of user interfaces for determining transducer locations for delivery of tumor treating fields using a subject selection tree.

FIG. 12 depicts an example of a subject selection tree according to an embodiment.

FIG. 13 depicts an example of an apparatus for determining transducers locations for delivery of TTFields based on external measurements of a subject according to an embodiment.

FIG. 14 depicts an example of an apparatus for determining transducer locations for delivery of TTFields using category information and a subject selection tree according to an embodiment.

FIG. 15 depicts an example apparatus to apply alternating electric fields to a subject's body.

FIGS. 16A-16B illustrate schematic views of exemplary design of a transducer for applying alternating electric fields.

FIG. 17 depicts an example placement of transducers on a subject's head.

FIG. 18 depicts an example computer apparatus.

Various embodiments are described in detail below with reference to the accompanying drawings, wherein like reference numerals represent like elements.

### DESCRIPTION OF EMBODIMENTS

When generating transducer layouts for application of tumor treating fields (TTFields) for a subject, medical images of the subject are often used. Examples of such medical images are magnetic resonance imaging (MRI) scans, computed tomography (CT) medical image, positron emission tomography (PET) medical images, and/or the like including combinations and/or multiples thereof. Segmentation is often performed on medical images to divide a medical image into two or more segments with each segment representing a different object of interest. A medical image is often segmented to extract or isolate an object of interest (e.g., abnormal tissue) in the medical image from other structures (e.g., healthy tissue). As discovered by the inventors, performing segmentation on a medical image of a subject uses significant resources in terms of computing power and healthcare provider time. For example, to segment an image, the image is sliced, and an abnormality is segmented from healthy tissue in each slice, requiring significant time from the healthcare provider and significant computing resources to perform the segmentation. Moreover, segmentation on medical images of certain regions of the body are particularly computationally demanding. For example, medical images of a head typically show about five different types of tissues, while medical images of a torso show about twenty different types of tissues, making segmenting medical images of a torso significantly more computationally demanding than segmenting medical images of a head. Medical images of a torso are also significantly larger than medical images of a torso, for example, due to the relative size different in heads and torsos. Consequently, it may take a few minutes to segment a medical image of a head but upwards of an hour to segment a medical image of a torso.

One or more embodiments described herein provide for generating transducer layouts for application of TTFields for a subject without performing segmentation. In some embodiments, a healthy model may be selected from multiple healthy models, the selected healthy model being selected to represent a subject. For example, a healthy model with similar characteristics/properties (e.g., height, weight, body mass, gender, etc.) to the subject may be selected. For example, measurements of the healthy subject are taken from the medical image and compared to corresponding measurements in healthy models. A healthy model is then selected based on the measurements. The healthy model is then used to select locations to place transducers to treat the abnormality. A dosage of TTFields treatment is then calculated, for each of the locations, for a region of interest in the healthy model of the subject without modifying the healthy model to include abnormal tissue. Thus, a subject-specific transducer layout is generated for the subject without segmenting medical images of the subject by using the healthy models.

The embodiments described herein further provide a practical application of generating transducer layouts by avoiding the need to perform segmentation on medical images of the subject. By using the healthy models, the tissue conductivity is considered when generating transducer layouts for treating the subject with TTFields without having to perform segmentation on medical images of the subject. This approach yields more effective TTFields treatment for the subject while saving computational processing resources and healthcare provider time. These and other technical improvements may be realized using the one or more embodiments described herein.

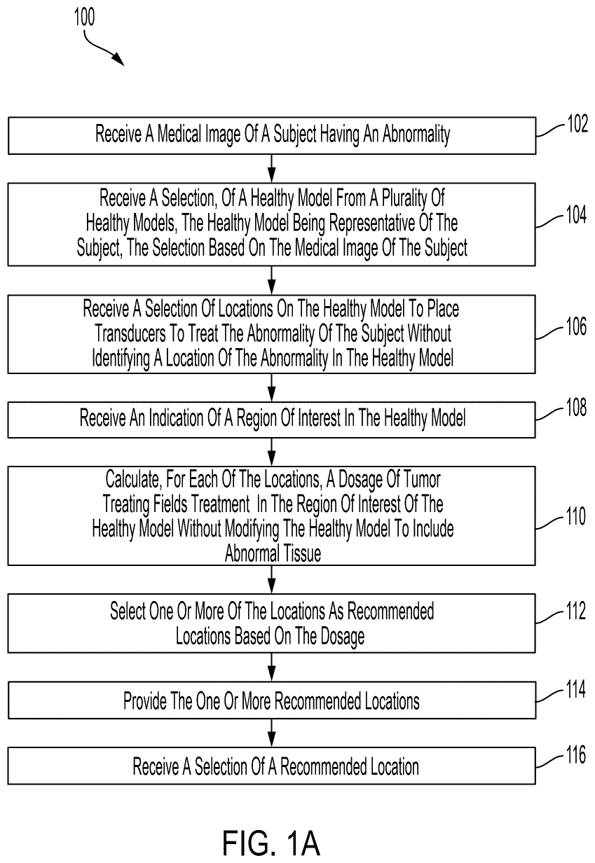

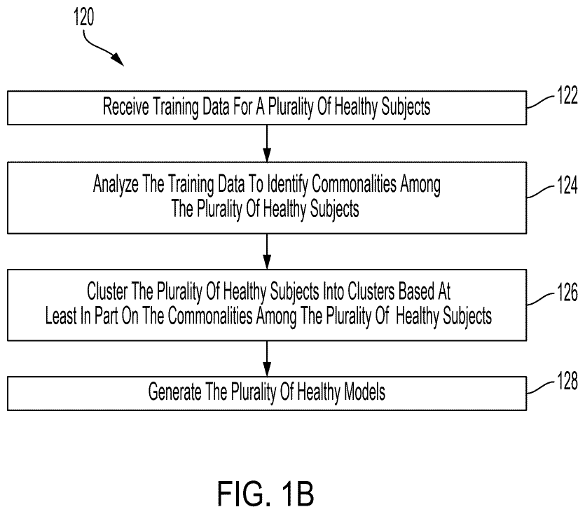

FIG. 1A depicts an example method for determining transducer locations for delivery of tumor treating fields using simulations based on models of healthy subjects. Certain steps of the method 100 are described as computer-implemented steps. The computer may be, for example, any device comprising one or more processors and memory accessible by the one or more processors, the memory storing instructions that when executed by the one or more processors cause the computer to perform the relevant steps of the method 100. The method 100 may be implemented by any suitable system or apparatus, such as the apparatus 1800 of FIG. 18. While an order of operations is indicated in FIG. 1A for illustrative purposes, the timing and ordering of such operations may vary where appropriate without negating the purpose and advantages of the examples set forth in detail herein.

With reference to FIG. 1A, at step 102, the method 100 includes receiving a medical image of a subject having an abnormality. According to one or more embodiments described herein, the abnormality is a tumor or another type of non-healthy tissue. According to one or more embodiments described herein, the medical image is a computed tomography (CT) image, a magnetic resonance imaging (MRI) medical image, a positron emission tomography (PET) medical image, and/or the like including combinations and/or multiples thereof. The medical image can be of all or a portion of the subject. For example, the medical image can be an MRI, CT, or PET image of a head of the subject, a torso of the subject, and/or another portion of the subject.

At step 104, the method 100 includes receiving a selection, of a healthy model from a plurality of healthy models, the healthy model being representative of the subject, the selection based on the medical image of the subject. In some embodiments, healthy models may be models of subjects known to be without or substantially without abnormalities in an area of interest (e.g., a head, torso, and/or the like including combinations and/or multiples thereof). For example, medical images of healthy subjects (e.g., those known to be without abnormalities in an area of interest) are captured and used to create healthy models of the healthy subjects. According to one or more embodiments described herein, the healthy model is selected from the plurality of healthy models based at least in part on a medical image of the subject (e.g., MRI image(s), CT image(s), PET image(s)), a physical measurement of the subject (e.g., a circumference of the subject's head or torso), a classification of the subject (e.g., a gender of the subject), and/or the like including combinations and/or multiples thereof.

According to one or more embodiments described herein, the plurality of healthy models is based on healthy subjects clustered into groups. For example, the healthy models can be grouped based on characteristics of the healthy subjects from with the healthy models were created. The healthy models can be grouped according to any suitable characteristic(s), such as weight of the subject, height of the subject, body mass of the subject, gender of the subject, health information about the subject (e.g., previously surgical procedures, known diseases), and/or the like including combinations and/or multiples thereof. According to one or more embodiments described herein, each of the plurality of healthy models is representative of a group of healthy subjects without abnormalities. According to one or more embodiments described herein, each of the plurality of healthy models is segmented based on tissue type. The selection of the healthy model can be determined automatically (e.g., based on analyzing one or more medical images of the subject to identify similarities to one of the heathy models), manually (e.g., a healthcare provider can select a healthy model from the plurality of healthy models), and/or the like including combinations and/or multiples thereof. According to one or more embodiments described herein, the selection of the healthy model is based on identifying a landmark in the medical image of the subject and a corresponding landmark in the healthy model. A landmark can be any suitable internal or external feature, such as an organ (e.g., heart, liver, lung, etc.), a bone, and/or the like including combinations and/or multiples thereof.

According to one or more embodiments described herein, the selection of the healthy model is based on measurements of the subject and measurements for the healthy models. For example, a healthy model is selected by determining a plurality of measurements of the subject from the medical image of the subject. Such measurements can be, for example, a circumference of the head or torso of the subject, length, depth, and/or width of the head or torso of the subject, a size of an organ (e.g., heart, liver, lung, etc.) of the subject, and/or the like including combinations and/or multiples thereof. The selection is further performed by comparing the plurality of measurements of the subject to a plurality of measurements for each healthy model. For example, if measurements of the subject include length, width, and circumference of the torso of the subject, such measurements can be compared to corresponding measurements of healthy models. The healthy model is then selected to be representative of the subject based on the healthy model having measurements most similar to the measurements of the subject. According to one or more embodiments described herein, if the medical image of the subject is of a torso of the subject, the plurality of measurements of the subject may include at least five measurements of the subject. It should be appreciated that other numbers of measurements can be used (e.g., more than five or less than five). According to one or more embodiments described herein, the healthy model is selected based on a location of an organ of the subject. That is, a location of an organ (e.g., heart, liver, lung, etc.) of the subject is compared to a corresponding to location of the same organ in the healthy models, and a healthy model is selected based on the location of the organ being similar between the subject and the heathy model.

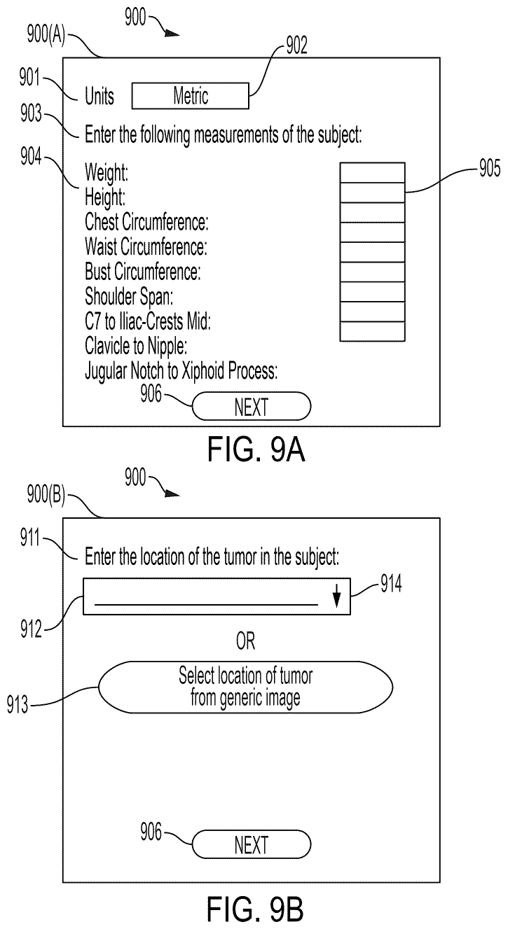

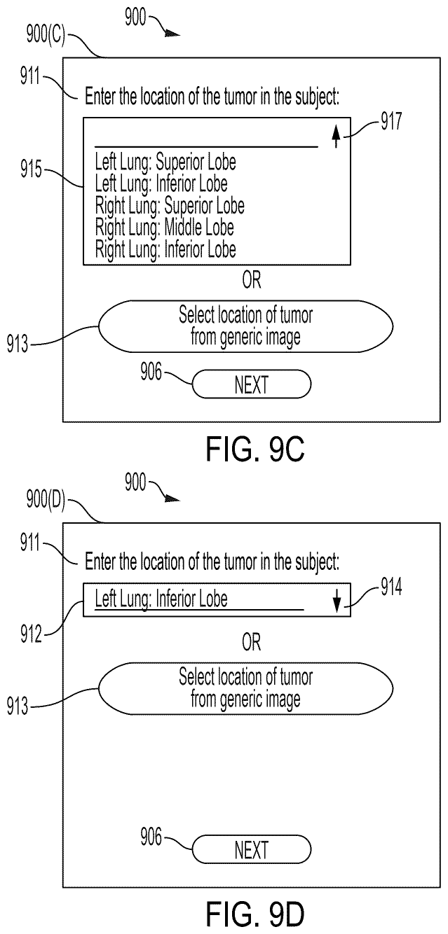

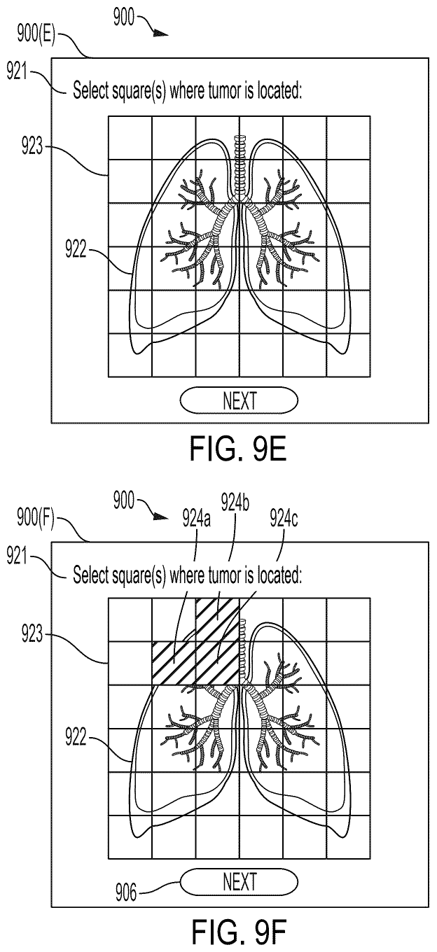

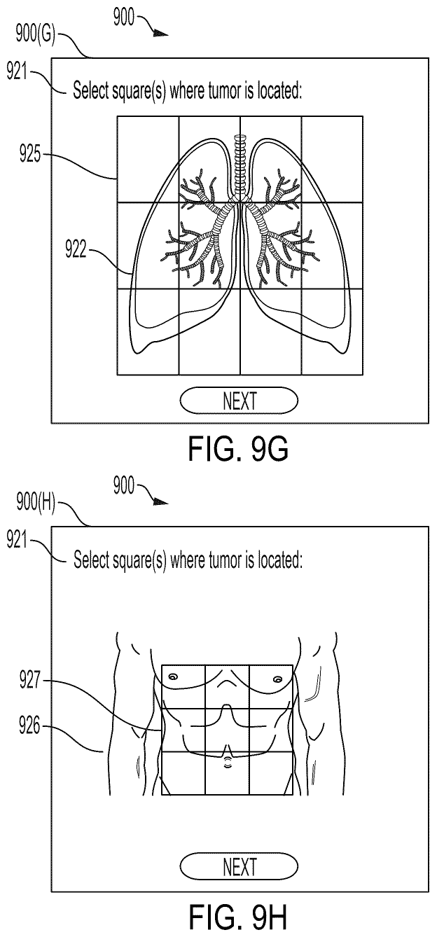

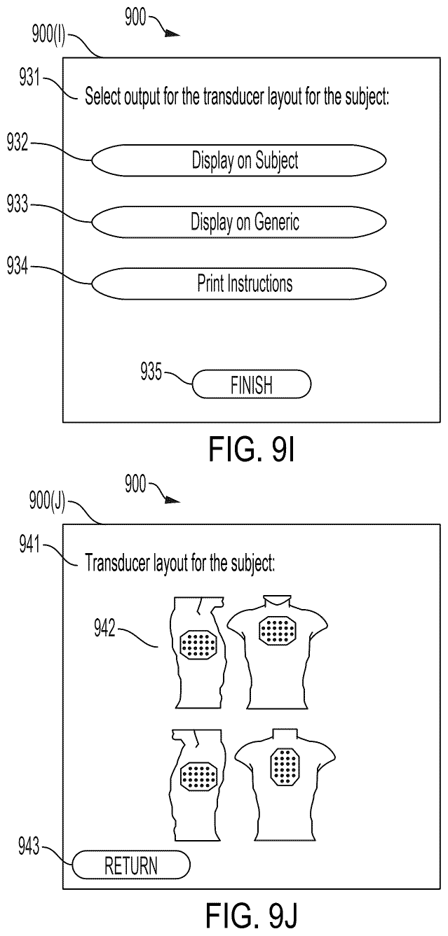

According to one or more embodiments described herein, the healthy models are generated according to the method 120 of FIG. 1B, which is now described in more detail. In particular, FIG. 1B depicts an example method 120 for generating healthy models according to an embodiment. Certain steps of the method 120 are described as computer-implemented steps. The computer may be, for example, any device comprising one or more processors and memory accessible by the one or more processors, the memory storing instructions that when executed by the one or more processors cause the computer to perform the relevant steps of the method 120. The method 120 may be implemented by any suitable system or apparatus, such as the apparatus 900 of FIG. 9. While an order of operations is indicated in FIG. 1B for illustrative purposes, the timing and ordering of such operations may vary where appropriate without negating the purpose and advantages of the examples set forth in detail herein.

At step 122, the method 120 includes receiving training data for a plurality of healthy subjects. The training data includes information about the healthy subjects. For example, the training data can include medical history information (e.g., past diagnoses, past treatments), demographic information (e.g., height, weight, gender, body mass), medical images (e.g., MRI images, CT images), and/or the like including combinations and/or multiples thereof.

At step 124, the method 120 includes analyzing the training data to identify commonalities among the plurality of healthy subjects. Examples of commonalities include height, weight, gender, body mass, and/or the like including combinations and/or multiples thereof. According to one or more embodiments described herein, analyzing the training data includes performing a principal component analysis (PCA) to identify the commonalities among the plurality of healthy subjects. In other embodiments, other approaches to performing the commonality analysis can be performed.

At step 126, the method 120 includes clustering the plurality of healthy subjects into clusters based at least in part on the commonalities among the plurality of healthy subjects. For example, subjects that are similar in height, weight, gender, and body mass may be clustered into a cluster. According to one or more embodiments described herein, the clustering is performed using k-means clustering. In other embodiments, other approaches to cluster can be performed.

At step 128, the method 120 includes generating the plurality of healthy models. Generating the healthy models includes, for each cluster, generating one of the plurality of healthy models based at least in part on the training data for the plurality of healthy subjects that are within the cluster. According to an embodiment, a model of one of the plurality of healthy subjects within the cluster is selected to be the healthy model. According to another embodiment, the healthy model for the cluster is generated using information from two or more of the healthy subjects within the cluster. According to one or more embodiments described herein, each of the healthy models is defined by a vector having a number of variables. The healthy model can be created from the variables of the healthy models of the healthy subjects within the cluster. As an example, the healthy model is an average of the variables of healthy models within the cluster.

According to one or more embodiments described herein, generating the plurality of healthy models includes selecting, for each cluster, a model of one of the healthy subjects within the cluster to be the healthy model. According to one or more embodiments described herein, generating the plurality of healthy models includes generating, for each cluster, the healthy model using information about at least two of the healthy subjects within the cluster. For example, the healthy model can be generated by averaging values from two or more healthy subjects within the cluster. According to one or more embodiments described herein, each of the healthy subjects has a medical image associated therewith, and measurements for each healthy subject are extracted from the medical image associated with each healthy subject. The measurements are used to select a healthy model as described with reference to step 104, for example.

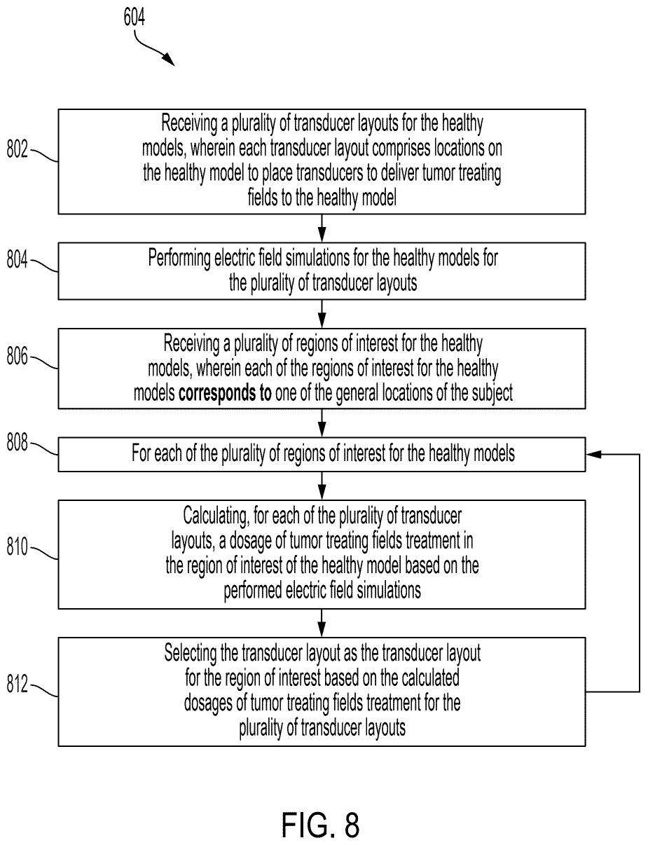

With continued reference to FIG. 1A, at step 106, the method 100 includes receiving a selection of locations on the healthy model to place transducers to treat the abnormality. According to one or more embodiments described herein, the selection of locations on the healthy model to place the transducers to treat the abnormality is based on the location of the abnormality with respect to the subject. As an example, if the abnormality is in the head of the subject, one or more of locations on the healthy model may include a first pair of transducers with a front-back relationship and a second pair of transducers with a left-right relationship (such as the transducer layout in FIG. 8). As an example, if the abnormality is in the torso of the subject, one or more of locations on the healthy model may include a first pair of transducers with a front-back relationship and a second pair of transducers with a left-right relationship. According to one or more embodiments described herein, the selection of locations on the healthy model to place the transducers to treat the abnormality is based at least in part on a preselected list of locations based on, for example, the healthy model and a general location of an abnormality in the subject (e.g., in the center of the head, in the front of the head, in the front left of the head, in the upper torso, in the upper left torso, etc.). The locations on the healthy model can be selected automatically (e.g., based on analyzing one or more medical images of the subject), manually (e.g., a healthcare provider can select the locations), and/or the like including combinations and/or multiples thereof.

At step 108, the method 100 includes receiving an indication of a region of interest (ROI) in the healthy model. According to one or more embodiments described herein, the region of interest is defined by a gross tumor volume (GTV) for the abnormality, which is then used to calculate the dosage of treatment (at step 112). The indication of the ROI can be determined automatically (e.g., based on analyzing one or more medical images of the subject to determine the ROI), manually (e.g., a healthcare provider can indicate the ROI), and/or the like including combinations and/or multiples thereof.

At step 110, the method 100 includes calculating, by at least one processor (e.g., the one or more processors 902 of FIG. 9), for each of the plurality of locations (from step 106), a dosage of TTFields treatment for the region of interest in the healthy model without modifying the healthy model to include abnormal tissue. According to one or more embodiments described herein, the healthy model defines healthy tissue having an electrical property, and calculating the dosage of TTFields treatment is based at least in part on the electrical property. For example, the healthy model can define a conductivity of one or more types of healthy tissue, and the dosage of TTFields treatment may be calculated using the conductivity for each of the one or more types of healthy tissue. Calculating the dosage of TTFields treatment is further described in more detail in U.S. Patent Application Publication No. 2020/0023179, entitled “USING POWER LOSS DENSITY AND RELATED MEASURES TO QUANTIFY THE DOSE OF TUMOR TREATING FIELDS (TTFIELDS)” and U.S. Patent Application Publication No. 2021/0196943, entitled “METHODS, SYSTEMS, AND APPARATUSES FOR FAST APPROXIMATION OF ELECTRIC FIELD DISTRIBUTION,” the entire contents of both of which are incorporated by reference herein. It should be appreciated that these calculations in step 110 involve solving complex algorithms using large data sets associated with the healthy model and associated with the subject and, as such, require the use of a computer apparatus, as the human mind is not capable of performing the required calculations.

At step 112, the method 100 includes selecting one or more of the locations as recommended locations based on the dosage calculated in step 110. According to one or more embodiments described herein, the location with a highest dosage for the ROI may be selected as a recommended location. According to one or more embodiments described herein, the location with a second highest dosage for the ROI may be selected as a recommended location. According to one or more embodiments described herein, the location with a highest dosage for the ROI that also matches other criteria may be selected as a recommended location. As an example, other criteria to be considered may include: avoiding one or more avoidance areas of the subject (e.g., a nipple, a surgical scar, an eye, an ear, a mouth, a nose, irritated skin, a sensitive area, etc.); and providing maximized patient comfort. The recommended locations can be selected automatically (e.g., based on analyzing the dosages and the locations), manually (e.g., a healthcare provider can analyze the dosages and the locations), and/or the like including combinations and/or multiples thereof.

At step 114, the method 100 includes providing the one or more recommended locations selected in step 112. According to one or more embodiments described herein, each recommended location may be output as a representation of one or more pairs of transducers and locations for the one or more pairs of transducers on the subject. As an example, the representation of the subject may be based on the healthy model or the subject. As an example, a display is used to show a representation of the transducers and/or the locations on the subject. As an example, the output of the representation may be in form of a document.

At step 116, the method 100 includes receiving a selection of a recommended location. The recommended location can be selected automatically (e.g., based on analyzing one or more medical images of the subject), manually (e.g., a healthcare provider can select the recommended location from the possible locations), and/or the like including combinations and/or multiples thereof. The selected location can then be used to provide the dosage of TTFields treatment to the subject.

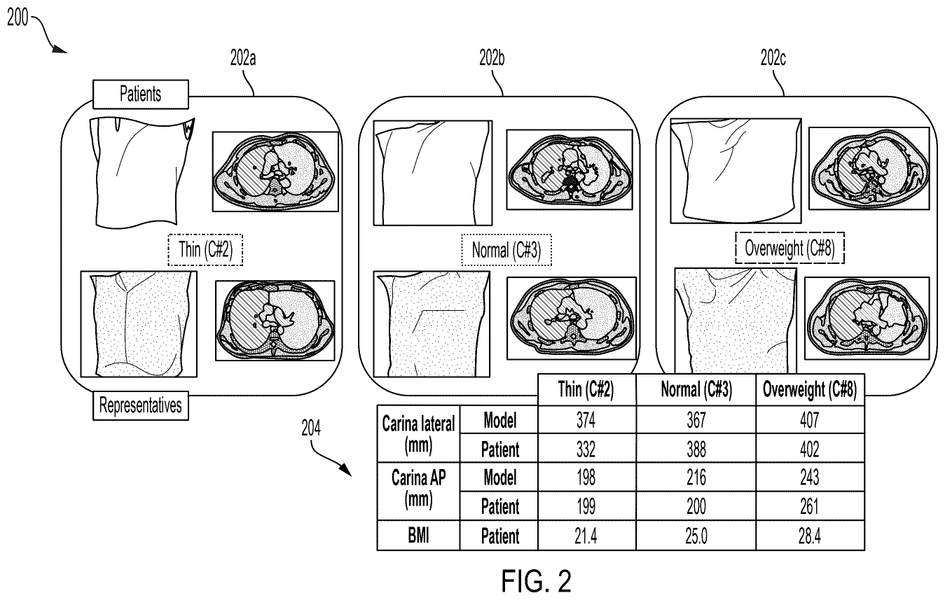

FIG. 2 depicts examples of healthy models 202a-202c. In this example, the healthy model 202a is a healthy model for a “thin” classification, the healthy model 202b is a healthy model for a “normal” classification, and the healthy model 202c is a healthy model for an “overweight” classification. As shown in the table 204, various values are associated with each of the healthy models 202a-202c, such as values for a carina lateral measurement, a carina AP measurement, and a body mass index. It should be appreciated that additional and/or alternative values can be associated with one or more of the healthy models 202a-202c in other embodiments. As can be seen in the table 204, corresponding values for the subject (e.g., “Patient”) are also shown.

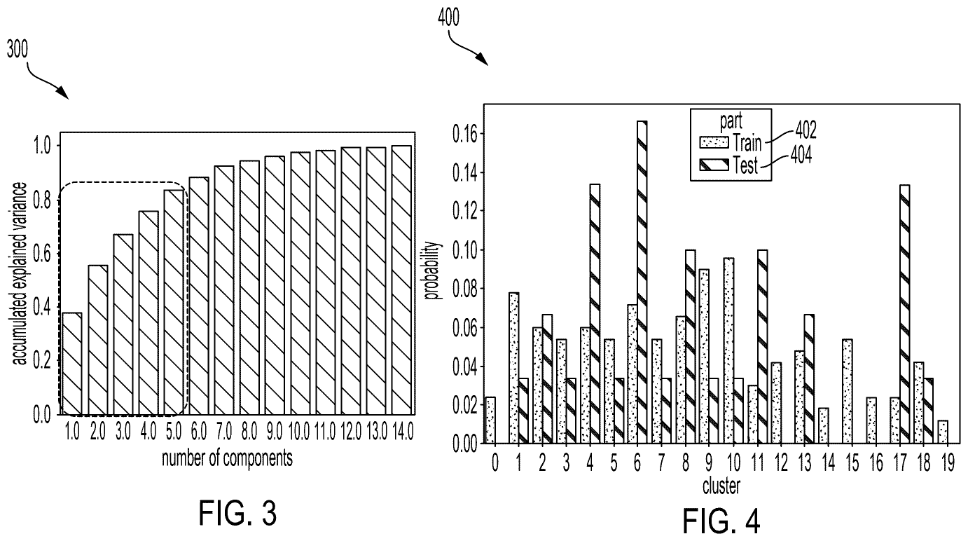

FIG. 3 depicts an example plot 300 used for clustering healthy models. In this example, a number of components are identified and plotted based on their accumulated explained variance. The components can be identified automatically (e.g., by a machine learning model trained to identify components from data of subjects), manually (e.g., selected by a subject matter expert), and/or combinations thereof. Non-limiting examples of components are: a carina anteroposterior (AP) measurement, a carina lateral measurement, a carina thoracic cavity lateral measurement a carina thoracic cavity lateral AP measurement, a carina mediastinum width measurement, a top liver AP measurement, a top liver lateral measurement, a top liver thoracic cavity lateral measurement, a top liver thoracic cavity AP measurement, a suprasternal notch AP measurement, a suprasternal notch lateral measurement, a suprasternal notch thoracic cavity lateral measurement, a suprasternal notch thoracic cavity AP measurement, a suprasternal notch to top liver measurement, and/or the like including combinations and/or multiples thereof. Principal component analysis can be used to identify a certain number of features as being relevant. For example, although fourteen components are used in the example of FIG. 3, it can be shown that the first five components account for 80% of the data and thus the remaining components can be ignored or discarded according to one or more embodiments described herein. In such cases, each healthy model would have a vector of five variables, each of the five variables corresponding to one of the five components.

FIG. 4 depicts an example plot 400 used for clustering healthy models. In this example, training data 402 and test data 404 are plotted for each of twenty clusters based on their respective probabilities. The training data 402 represents data about subjects that are used to train a machine learning model, for example, to perform clustering, and the test data 404 represents data about subjects used to test or verify the machine learning model after training. It should be appreciated that the training data 402 and the testing data 404 are different data according to one or more embodiments described herein.

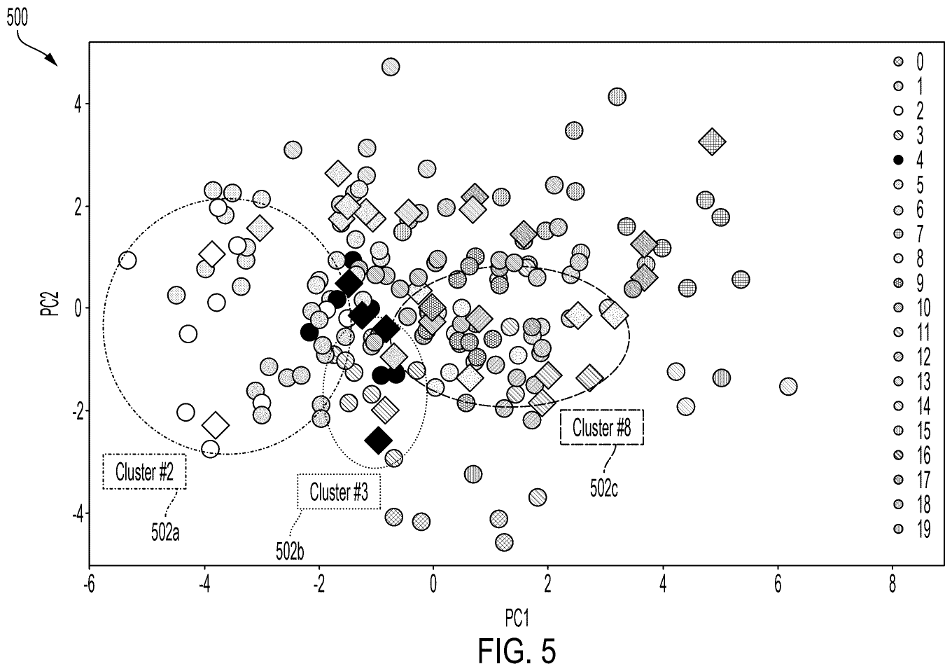

FIG. 5 depicts an example plot 500 of clusters of healthy models. In this example, data points for twenty different clusters are shown, corresponding to the plot 400. Three clusters 502a, 502b, 502c are shown, which correspond to the heathy models 202a, 202b, 202c, respectively, of FIG. 2. In the example of FIG. 5, data points for each cluster fall within a geometric shape, such as a circle or ellipse, as shown. According to one or more embodiments described herein, a centroid of the shape can be determined and can be used as the healthy model for that cluster. According to one or more embodiments described herein, one of the data points within the cluster can be selected to be the healthy model of the cluster.

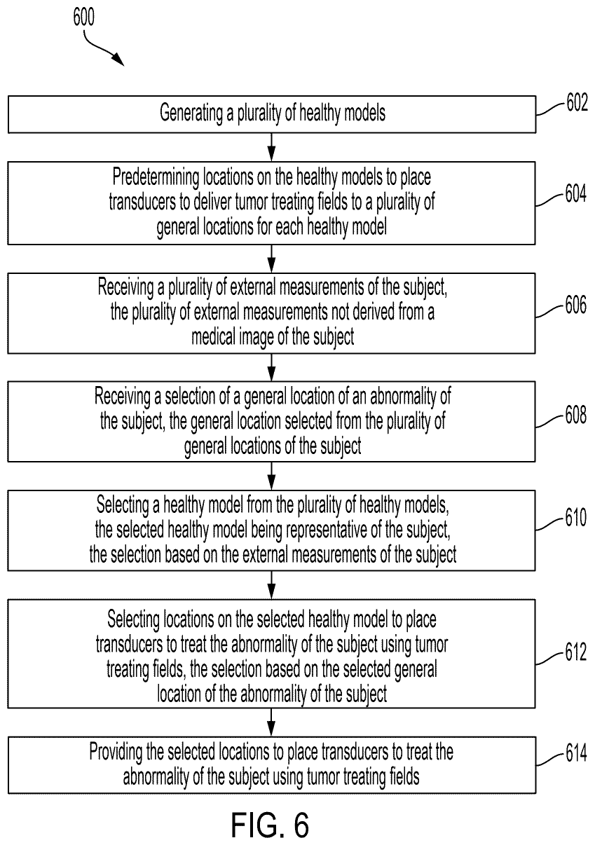

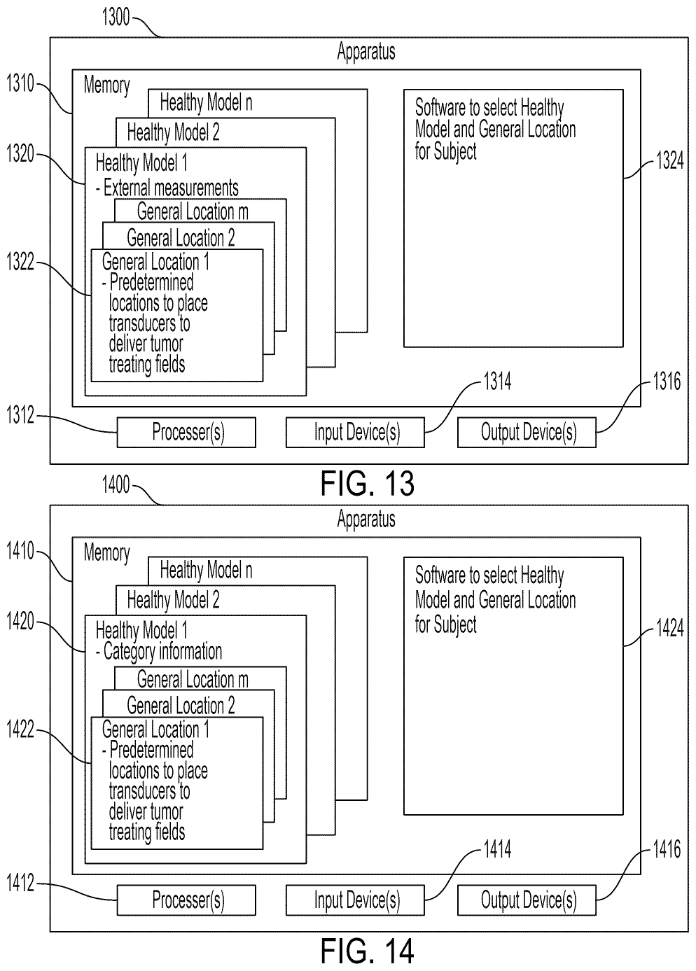

FIG. 6 depicts an example method of determining transducer locations for delivery of TTFields based on external measurements of the subject. Certain steps of the method 600 are described as computer-implemented steps. The computer may be, for example, any device comprising one or more processors and memory accessible by the one or more processors, the memory storing instructions that when executed by the one or more processors cause the computer to perform the relevant steps of the method 600. The method 600 may be implemented by any suitable system or apparatus, such as apparatus 1300 of FIG. 13 or apparatus 1800 of FIG. 18. While an order of operations is indicated in FIG. 6 for illustrative purposes, the timing and ordering of such operations may vary where appropriate without negating the purpose and advantages of the examples set forth in detail herein.

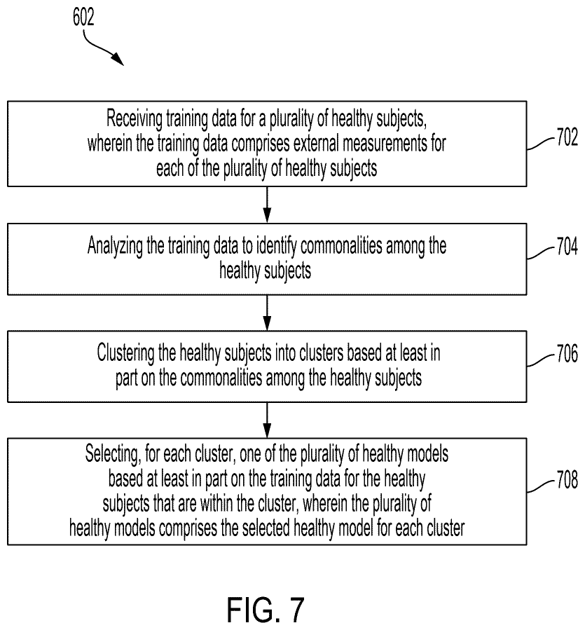

At step 602, the method 600 may include generating a plurality of healthy models bard on a plurality of healthy subjects. More details of the generating in step 602 will be described in FIG. 7 and its accompanying description.

At step 604, the method 600 may include predetermining locations on the healthy models to place transducers to deliver TTFields to a plurality of general locations for each healthy model. In some embodiments, the predetermination may be performed prior to receiving the external measurements of the subject. More details of the predetermining in step 604 will be described in FIG. 8 and its accompanying description.

In some embodiments, predetermining locations on the healthy models to place transducers may be based at least in part on conductivities for at least one tissue type included in the healthy models. In some embodiments, the healthy models may define healthy tissues having electrical properties and predetermining locations on the healthy models to place transducers may be based on at least in part on the electrical properties.

In some embodiments, the plurality of general locations may be in a torso, a thorax, or an abdomen of the subject. In some embodiments, the plurality of general locations may include clinically significant regions of an organ of the subject. For example, the plurality of general locations may include a superior lobe in a left lung, an inferior lobe in the left lung, a superior lobe in a right lung, a middle lobe in the right lung, and an inferior lobe in the right lung.

In some embodiments, the plurality of general locations may include areas divided by a grid of a region of the subject where the abnormality of the subject is located. For example, assuming the plurality of general locations are identified in lungs of the subject, the plurality of general locations may include an upper area, a middle area, and a lower area in a left lung and an upper area, a middle area, and a lower area in a right lung. As another example for the lungs, the plurality of general locations may include a grid of six areas in a left lung and a grid of six areas in a right lung (such as the example shown in FIG. 9G). As another example, the plurality of general locations may include a grid of nine areas in an abdomen of the subject (such as the example shown in FIG. 9H).

At step 606, the method 600 may include receiving a plurality of external measurements of the subject, where the plurality of external measurements are not derived from a medical image of the subject. In some embodiments, the plurality of external measurements may be obtained from a physical measurement of the subject. For example, the external measurements of the subject may include at least one of the following measurements: a weight of the subject (e.g., kilogram (kg)); a height of the subject (e.g., centimeter (cm)); a chest circumference of the subject (e.g., cm); a waist circumference of the subject (e.g., cm); a bust circumference of the subject (e.g., cm); a shoulder span of the subject (e.g., cm); a measurement from C7 to iliac-crests mid (e.g., cm); a measurement from the clavicle to a nipple of the subject (e.g., cm); and a measurement from the jugular notch to the xiphoid process (e.g., cm). “C7” refers to the seventh cervical vertebra, a bony bump located at the base of a person's neck, which is used as a reference point when measuring a torso length from a neck to a hip bone (iliac crest). “C7 to iliac crest” generally may indicate a distance from a prominent bone at the base of the neck to the top of the hip bone. For example, the external measurements of the subject may include at least two, three, four, five, six, seven, eight, or nine of the above measurements. In some embodiments, the external measurements may be supplemented with the gender and/or age (e.g., years) of the subject.

In some embodiments, the plurality of healthy models may be generated in step 602 prior to receiving the plurality of external measurements of the subject in in step 606.

In some embodiments, the external measurements of the subject may be received from a user interface for a user to enter the external measurements of the subject. In some embodiments, the external measurements of the subject may be calculated using an application (app) on a personal computing device, wherein the app calculates the external measurements of the subject based on images of the subject obtained with a camera on the personal computing device. In some embodiments, the external measurements of the subject may be received from the app on the personal computing device. More details in this regard will be described in FIGS. 9A-9J and their accompanying descriptions.

At step 608, the method 600 may include receiving a selection of a general location of an abnormality of the subject, where the general location is selected from the plurality of general locations of the subject. In some embodiments, the general location of the abnormality of the subject may be received from a user interface providing at least one of a list or an image of the plurality of general locations of the subject, wherein the general location may be selectable by a user from the list or the image.

At step 610, the method 600 may include selecting a healthy model from the plurality of healthy models, where the selected healthy model is representative of the subject, where the selection is based on the external measurements of the subject. In some embodiments, selecting the healthy model may include comparing the external measurements of the subject to external measurements for each of the plurality of healthy models and selecting the healthy model being representative of the subject as the healthy model having external measurements most similar to the external measurements of the subject. In particular, each of the plurality of healthy models may be representative of a group of healthy subjects without abnormalities.

In some embodiments, the plurality of healthy models may be based on models of healthy subjects clustered into groups according to external measurements of the healthy subjects. For example, each of the healthy models may be defined by a vector having a number of variables corresponding to external measurements for the healthy model.

At step 612, the method 600 may include selecting locations on the selected healthy model to place transducers to treat the abnormality of the subject using tumor treating fields, where the selection is based on the selected general location of the abnormality of the subject.

At step 614, the method 600 may include providing the selected locations to place transducers to treat the abnormality of the subject using tumor treating fields. In some embodiments, the selected locations to place transducers to treat the abnormality of the subject using tumor treating fields may be the corresponding predetermined locations on the selected healthy model for the selected general location.

In some embodiments, providing the selected locations to place transducers may include at least one of: presenting an image of the subject with identifications of the selected locations to place transducers; presenting an image of a generic subject with identifications of the selected locations to place transducers; or presenting a description of the selected locations to place transducers.

FIG. 7 depicts an example method of generating the plurality of healthy models in step 602 in FIG. 6.

At step 702, the method of step 602 may include receiving training data for a plurality of healthy subjects, wherein the training data comprises external measurements for each of the plurality of healthy subjects. In some embodiments, each of the healthy subjects includes at least one medical image associated therewith, wherein external measurements for the healthy subjects are not derived from the medical images associated with the healthy subjects.

At step 704, the method of step 602 may include analyzing the training data to identify commonalities among the healthy subjects. In some embodiments, analyzing the training data may include performing a principal component analysis to identify the commonalities among the plurality of healthy subjects. In particular, the commonalities may be based on principal components defined for each of the healthy subjects. As one example, the principal components may be defined by a subject matter expert. As another example, the principal components may be defined automatically based on analyzing the training data.

At step 706, the method of step 602 may include clustering the healthy subjects into clusters based at least in part on the commonalities among the healthy subjects. In some embodiments, the clustering may be performed using k-means clustering.

At step 708, the method of step 602 may include selecting, for each cluster, one of the plurality of healthy models based at least in part on the training data for the healthy subjects that are within the cluster, wherein the plurality of healthy models may comprise the selected healthy model for each cluster.

In some embodiments, selecting, for each cluster, one of the plurality of healthy models may include: determining a center in n-dimensional space for each cluster, where each cluster is defined by an n-dimensional vector, where the dimensions of the n-dimensional vector correspond to the external measurements; and selecting the model of the healthy subjects in the cluster closest to the center of the cluster as the healthy model for the cluster. In some embodiments, selecting, for each cluster, one of the plurality of healthy models may include generating, for each cluster, the healthy model using information regarding at least two of the healthy subjects within the cluster.

FIG. 8 depicts an example method of predetermining locations on the healthy models to place transducers to deliver tumor treating fields to the plurality of generated locations for each healthy model in step 604 in FIG. 6.

At step 802, the method of step 604 may include receiving a plurality of transducer layouts for the healthy models, where each transducer layout may include locations on the healthy model to place transducers to deliver tumor treating fields to the healthy model.

At step 804, the method of step 604 may include performing electric field simulations for the healthy models for the plurality of transducer layouts.

At step 806, the method of step 604 may include receiving a plurality of regions of interest for the healthy models, where each of the regions of interest for the healthy models may correspond to one of the general locations of the subject.

Steps 810 and 812 may be performed repetitively for each of the plurality of regions of interest for the healthy models at step 808. At step 810, the method of step 604 may include calculating, for each of the plurality of transducer layouts, a dosage of tumor treating fields treatment in the region of interest of the healthy model based on the performed electric field simulations. In some embodiments, calculating the dosage of tumor treating fields treatment in the region of interest of the healthy model may include averaging electric field strength for voxels of the medical image in the region of interest.

At step 812, the method of step 604 may include selecting the transducer layout as the transducer layout for the region of interest based on the calculated dosages of tumor treating fields treatment for the plurality of transducer layouts.

FIGS. 9A-9J depict examples of user interfaces for determining transducer locations for delivery of TTFields according to the method 600.

In FIG. 9A, a user interface 900 may present an initial window 900(A), showing units 901 and a drop list 902 of various units for height or size, an indication 903 asking the user to enter measurements of the subject, and indications 904 asking for various measurements. The use may enter the asked measurements in boxes 905. Upon entering the requested measurements, the user may click on “NEXT” button 906.

In FIG. 9B, the user interface 900 may present a window 900(B), showing an indication 911 asking the use to enter the location of the tumor in the subject. The use may select the location to be entered at blank 912 from a drop list by clicking on arrow 914, or by clicking on button 913 to select location of the tumor from generic image.

FIG. 9C, shows the user interface 900 if the user clicks on the arrow 914. In FIG. 9C, window 900(C) shows a drop list 915 including locations to be chosen from. After making a selection, the user may click on arrow 917 to close the drop list 915.

In FIG. 9D, window 900(D) shows a result upon the user selecting “Left Lung: Inferior Lobe” from the drop list 915 to be entered at the blank 912. The user may clink on the “NEXT” button 906 upon selecting a location from the drop list 915.

In FIG. 9E, the user interface shows window 900(E) upon the user click on 913 to select location of tumor from generic image. 900(E) displays an image 922 representing general locations of lungs of the subject and an indication 921 asking the user to select square(s) where tumor is location. As shown in FIG. 9E, the representative lungs 922 include areas divided by a grid of 36 squares 923, including a grid of eighteen areas in a left lung area and a grid of eighteen areas in a right lung area. The number of squares of the grid is not limited to 36, and may be more or less. As another example shown in FIG. 9G, window 900(G) displays that the representative lungs 922 are divided to 12 squares 925, including a grid of six areas in a left lung and a grid of six areas in a right lung. As another example shown in FIG. 9H, window 900(H) displays a FIG. 926 representing general location of an abdomen of the subject including a gird of nine areas 927.

FIG. 9F shows window 900(F), where the user selects three areas 924a, 924b, and 924c, representing location of the tumor. Upon the selection, the user may click on the “NEXT” button 906.

FIG. 9I shows window 900(I) upon the user entering the location of the tumor in the subject. 900(I) displays instructions 931 asking the user to select output for the transducer layout for the subject: display on an image of the subject 932, display on a generic model 933, and/or print instructions 934. 900(I) may be displayed upon selecting areas of location of the tumor. Upon making selection in window 900(I), the user may click on the “FINISH” button 935 to finish the selection to obtain a final result.

FIG. 9J shows window 900(J) displaying transducer layout 941 on a generic model, upon the user selecting 933 in window 900(I). Image 942 shows locations to place the transducer on the generic model. The use may click on “RETURN” button 943 to return to previous window(s) for alternative selections.

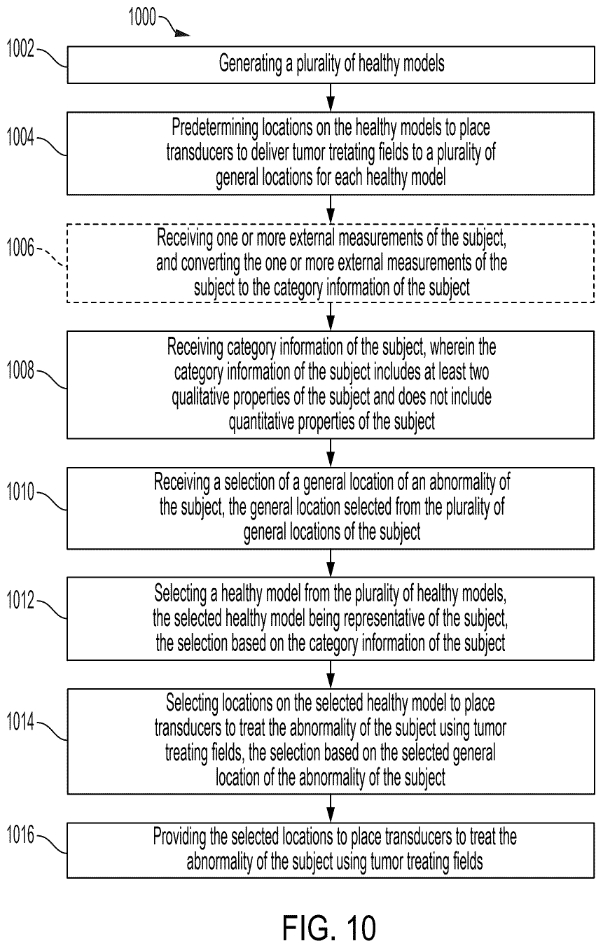

FIG. 10 depicts a method 1000 for determining transducer locations for delivery of tumor treating fields using category information and a subject selection tree. Certain steps of the method 1000 are described as computer-implemented steps. The computer may be, for example, any device comprising one or more processors and memory accessible by the one or more processors, the memory storing instructions that when executed by the one or more processors cause the computer to perform the relevant steps of the method 1000. The method 1000 may be implemented by any suitable system or apparatus, such as apparatus 1300 of FIG. 13 or apparatus 1800 of FIG. 18. While an order of operations is indicated in FIG. 10 for illustrative purposes, the timing and ordering of such operations may vary where appropriate without negating the purpose and advantages of the examples set forth in detail herein.

At step 1002, the method 1000 may include generating a plurality of healthy models. In some embodiments, the step 1002 may be similar to the step 602 described in FIGS. 6 and 7. Specifically, generating the plurality of healthy models may include: receiving training data for a plurality of healthy subjects, where the training data may comprise category information for each of the plurality of healthy subjects; analyzing the training data to identify commonalities among the healthy subjects; clustering the healthy subjects into clusters based at least in part on the commonalities among the healthy subjects; and selecting, for each cluster, one of the plurality of healthy models based at least in part on the training data for the healthy subjects that are within the cluster, wherein the plurality of healthy models comprises the selected healthy model for each cluster.

In some embodiments, selecting, for each cluster, one of the plurality of healthy models may include: determining a center in n-dimensional space for each cluster, wherein each cluster is defined by an n-dimensional vector, where the dimensions of the n-dimensional vector may correspond to the category information; and selecting the model of the healthy subjects in the cluster closest to the center of the cluster as the healthy model for the cluster. In some embodiments, selecting, for each cluster, one of the plurality of healthy models may include generating, for each cluster, the healthy model using information regarding at least two of the healthy subjects within the cluster. In particular, each of the healthy subjects may have at least one medical image associated therewith, wherein category information for the healthy subjects is not derived from the medical images associated with the healthy subjects. The clustering may be performed using k-means clustering.

In some embodiments, analyzing the training data may include performing a principal component analysis to identify the commonalities among the plurality of healthy subjects. The commonalities may be based on principal components defined for each of the healthy subjects. The principal components may be defined by a subject matter expert. The principal components may be defined automatically based on analyzing the training data.

In particular, in method 1000, each of the healthy subjects may have at least one medical image associated therewith, where category information for the healthy subjects is not derived from the medical images associated with the healthy subjects.

At step 1004, the method 1000 may include predetermining locations on the healthy models to place transducers to deliver tumor treating fields to a plurality of general locations for each healthy model. In some embodiments, the step 1004 may be same as the step 604 described in FIGS. 6 and 8. In some embodiments, predetermining locations on the healthy models may be performed prior to receiving the category information of the subject.

At step 1006, the method 1000 may optionally include receiving one or more external measurements of the subject, and converting the one or more external measurements of the subject to the category information of the subject. For example, the one or more external measurements of the subject may include at least one of a height of the subject, a weight of the subject, or a body mass index of the subject.

In some embodiments, the external measurements of the subject may be received from a user interface for a user to enter the one or more external measurements of the subject. In some embodiments, the one or more external measurements of the subject may be calculated using an app on a personal computing device, where the app may calculate the one or more external measurements of the subject based on images of the subject obtained with a camera on the personal computing device. In some embodiments, the one or more external measurements of the subject may be received from the app on the personal computing device.

At step 1008, the method 1000 may include receiving the category information of the subject, wherein the category information of the subject includes at least two qualitative properties of the subject and does not include quantitative properties of the subject. In some embodiments, the plurality of healthy models may be generated prior to receiving the category information of the subject.

In some embodiments, the category information may include at least one of gender of the subject and size of the subject. In particular, the category information for the size of the subject may include a height of the subject and a girth of the subject. For example, the category information for the size of the subject may be selected from three options of small, medium, and large. For example, the category information for the height of the subject may be selected from three options, wherein the three options are short, medium, and tall. For example, the category information for the girth of the subject may be selected from three options, wherein the three options are thin, average, and heavy.

In some embodiments, the category information does not include external measurements of the subject. In some embodiments, the category information does not include internal measurements of the subject. In some embodiments, the category information does not include information derived from a medical image of the subject. In some embodiments, the category information of the subject may be received from a user interface for a user to enter the category information of the subject. The user interface may include a user-selectable option for gender of the subject, and the user interface may include a user-selectable option for a size of the subject.

At step 1010, the method 1000 may include receiving a selection of a general location of an abnormality of the subject, the general location selected from the plurality of general locations of the subject. The step 1010 may be same as step 608 in FIG. 6 and its accompanying description.

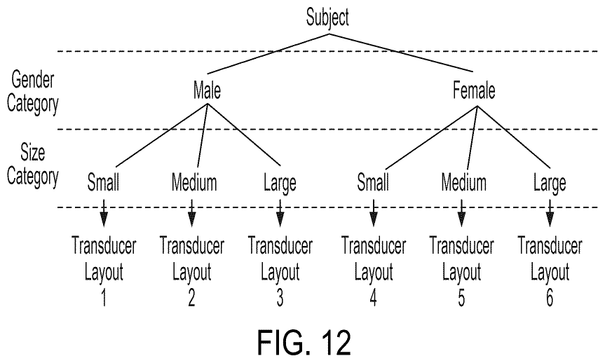

At step 1012, the method 1000 may include selecting a healthy model from the plurality of healthy models, the selected healthy model being representative of the subject, the selection based on the category information of the subject. In some embodiments, selecting the healthy model may be based on applying a selection tree to the category information of the subject. An example selection tree is depicted in FIG. 12 and discussed further below.

In some embodiments, selecting the healthy model may include comparing the category information of the subject to category information for each of the plurality of healthy models; and selecting the healthy model being representative of the subject as the healthy model having category information most similar to the category information of the subject.

At step 1014, the method 1000 may include selecting locations on the selected healthy model to place transducers to treat the abnormality of the subject using tumor treating fields, the selection based on the selected general location of the abnormality of the subject. In some embodiments, step 1014 may be same as the step 612 described in FIG. 6 and its accompanying description.

At step 1016, the method 1000 may include providing the selected locations to place transducers to treat the abnormality of the subject using tumor treating fields. In some embodiments, step 1016 may be same as the step 614 described in FIG. 6 and its accompanying description.

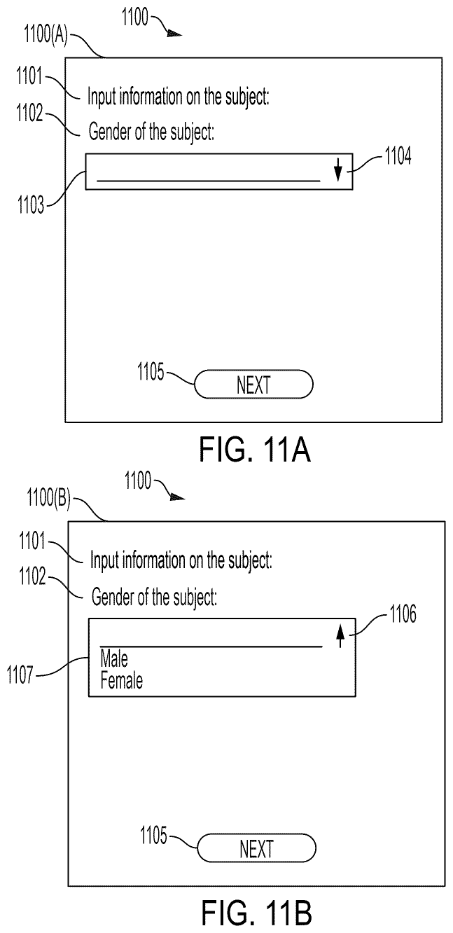

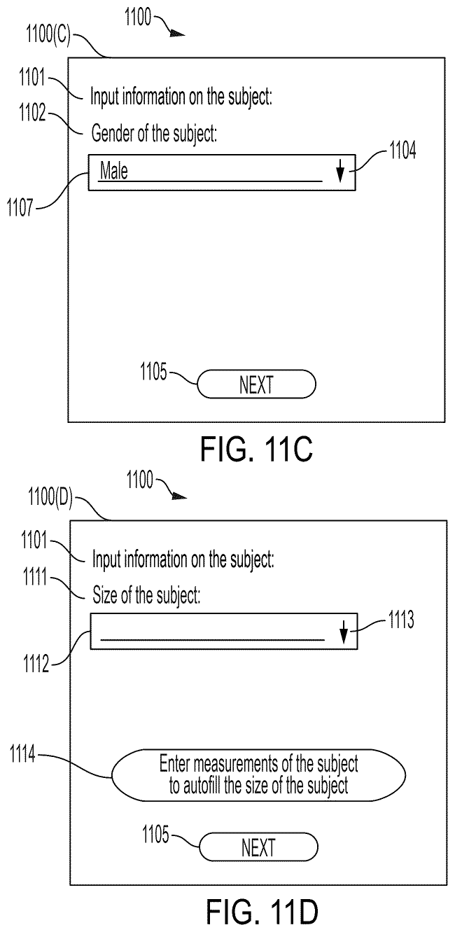

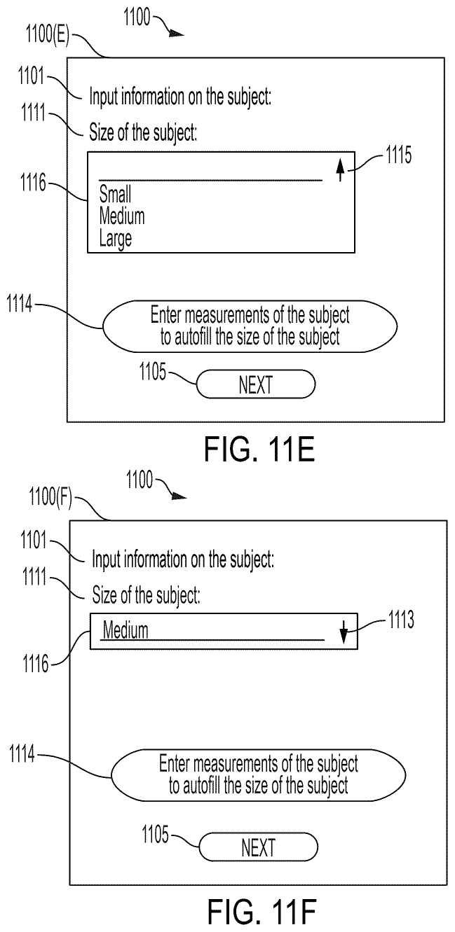

FIGS. 11A-11F depict examples of user interfaces for determining transducer locations for delivery of tumor treating fields using a subject selection tree.

In FIG. 11A, user interface 1100 shows an initial window 1100(A) including an indication 1101 asking the user to input information on the subject and indication 1102 asking the user to select gender of the subject. At block 1103, the user may click on arrow 1104 for a drop list.

FIG. 11B shows a window 1100(B) displaying drop list 1107 for the user to choose from. After selecting the gender of the subject, the user may click on arrow 1106 to close the drop list 1107.

FIG. 11C shows a window 1100(C) upon the user selecting the gender of the subject from the drop list 1107. In the present example, the user chooses “Male” as the gender of the subject. Upon making the selection, the user may click on “NEXT” button 1105.

In FIG. 11D, the user interface 1100 shows a window 1100(D) displaying an indication 1111 asking the user to select a size of the subject. At block 1112, the user may click on arrow 1113 for a drop list. The use may also click on indication 1114 to enter measurements of the subject to autofill the size of the subject.

FIG. 11E shows an example of the user interface 1100 if the user click on the arrow 1113. Window 1100(E) displays a drop list 1116 of size for the user to choose from. The user may click on arrow 1115 to close the drop list 1116.

FIG. 11F shows a window 1110(F) upon the user selects size of the subject from the drop list 1116. In the present example, the user chooses “Medium” as size of the subject. Upon making the selection, the user may click on “NEXT” button 1105.

FIG. 12 depicts an example of a subject selection tree according to an example. As shown in FIG. 12, combinations among selections of gender category (male or female) and size category (small, medium, or large) lead to six transducer layouts. These layouts may or may not be the same. The subject selection tree is not limited to the example in FIG. 12

FIG. 13 depicts an example of an apparatus for determining transducers locations for delivery of TTFields based on external measurements of a subject. Apparatus 1300 may include a memory 1310 including a plurality of healthy models 1 to n. The healthy models 1320 may be indexed by external measurements that differentiates the healthy models 1320 from each other. The external measurements of each healthy model may represent a corresponding representative group of healthy subjects. In particular, each of the plurality of healthy models 1320 may be indexed by general locations 1 to m for each healthy model, where each general location for each healthy model identifies predetermined locations 1322 on the healthy model to place transducers to deliver tumor treating fields to the general location.

The memory 1310 may also include a software 1324 to select a healthy model and a general location for the transducers for the subject.

The apparatus 1300 may also include one or more processors 1312, one or more input devices 1314, and one or more output devices 1316. The memory 1310 may be accessible by the one or more processors 1312.

The memory 1310 may store instructions that when executed by the one or more processors 1312, cause the apparatus 1300 to select a healthy model from the plurality of healthy models 1320, the selected healthy model being representative of a subject, the selection based on selected category information of the subject; select locations on the selected healthy model to place transducers to treat an abnormality of the subject using tumor treating fields, the selection based on a general location of the abnormality of the subject, the selection locations selected from the predetermined locations of the selected healthy model; and provide the selected locations to place transducers to treat the abnormality of the subject using tumor treating fields.

FIG. 14 depicts an example of an apparatus for determining transducer locations for delivery of TTFields using category information and a subject selection tree. Apparatus 1400 may include a memory 1410 including a plurality of healthy models 1 to n. The healthy models 1420 may be indexed by category information that differentiates the healthy models 1420 from each other. The category information of each healthy model may represent a corresponding representative group of healthy subjects. In particular, each of the plurality of healthy models 1420 may be indexed by general locations 1 to m for each healthy model, where each general location for each healthy model identifies predetermined locations 1422 on the healthy model to place transducers to deliver tumor treating fields to the general location.

The memory 1410 may also include a software 1424 to select a healthy model and a general location for the transducers for the subject.

The apparatus 1400 may also include one or more processors 1412, one or more input devices 1414, and one or more output devices 1416. The memory 1410 may be accessible by the one or more processors 1412.

The memory 1410 may store instructions that when executed by the one or more processors 1412, cause the apparatus 1400 to select a healthy model from the plurality of healthy models 1420, the selected healthy model being representative of a subject, the selection based on selected category information of the subject; select locations on the selected healthy model to place transducers to treat an abnormality of the subject using tumor treating fields, the selection based on a general location of the abnormality of the subject, the selection locations selected from the predetermined locations of the selected healthy model; and provide the selected locations to place transducers to treat the abnormality of the subject using tumor treating fields.

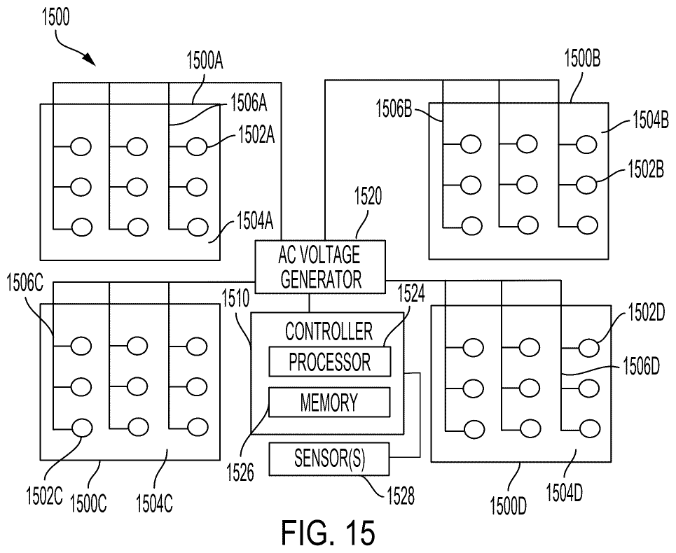

FIG. 15 depicts an example apparatus 1500 to apply alternating electric fields (e.g., TTFields) to the subject's body. The system may be used for treating a target region of a subject's body with an alternating electric field. According to one or more embodiments described herein, at least two transducers are used to provide alternating electric fields, such as TTFields, to a subject. For example, one electrode element or multiple electrode elements can be used to provide the alternating electric fields, such as TTFields, to the subject. According to one or more embodiments described herein, one or more pairs of transducers are used to deliver alternating electric fields, such as TTFields, to the subject. In an example, the target region may be in the subject's brain, and an alternating electric field may be delivered to the subject's body via transducers (e.g., a pair of transducers, two pairs of transducer arrays, and/or the like including combinations and/or multiples thereof) positioned on a head of the subject's body (such as, for example, in FIG. 17, which has four transducers 1700). In another example, the target region may be in the subject's torso, and an alternating electric field may be delivered to the subject's body via transducers (e.g., a pair of transducers, two pairs of transducer arrays, and/or the like including combinations and/or multiples thereof) positioned on at least one of a thorax, an abdomen, or one or both thighs of the subject's body. Other transducer array placements on the subject's body may be possible.

The example apparatus 1500 depicts an example system having four transducers (or “transducer arrays”) 1500A-D. It should be appreciated that other numbers of transducers can be used in other embodiments, such as two transducers or more than four transducers. Each transducer 1500A-D may include substantially flat electrode elements 1502A-D positioned on a substrate 1504A-D and electrically and physically connected (e.g., through conductive wiring 1506A-D). The substrates 1504A-D may include, for example, cloth, foam, flexible plastic, and/or conductive medical gel. Two transducers (e.g., 1500A and 1500D) may be a first pair of transducers configured to apply an alternating electric field to a target region of the subject's body. The other two transducers (e.g., 1500B and 1500C) may be a second pair of transducers configured to similarly apply an alternating electric field to the target region.

The transducers 1500A-D may be coupled to an AC voltage generator 1520, and the system may further include a controller 1510 communicatively coupled to the AC voltage generator 1520. The controller 1510 may include a computer having one or more processors 1524 and memory 1526 accessible by the one or more processors. The memory 1526 may store instructions that when executed by the one or more processors control the AC voltage generator 1520 to induce alternating electric fields between pairs of the transducers 1500A-D according to one or more voltage waveforms and/or cause the computer to perform one or more methods disclosed herein. The controller 1510 may monitor operations performed by the AC voltage generator 1520 (e.g., via the processor(s) 1524). One or more sensor(s) 1528 may be coupled to the controller 610 for providing measurement values or other information to the controller 1510.

In some embodiments, the voltage generation components may supply the transducers 1500A-D with an electrical signal having an alternating current waveform at frequencies in a range from about 50 kHz to about 1 MHz and appropriate to deliver TTFields treatment to the subject's body

The electrode elements 1502A-D may be capacitively coupled. In one example, the electrode elements 1502A-D are ceramic electrode elements coupled to each other via conductive wiring 1506A-D. When viewed in a direction perpendicular to its face, the ceramic electrode elements may be circular shaped or non-circular shaped. In other embodiments, the array of electrode elements are not capacitively coupled, and there is no dielectric material (such as ceramic, or high dielectric polymer layer) associated with the electrode elements.

The structure of the transducers 1500A-D may take many forms. The transducers may be affixed to the subject's body or attached to or incorporated in clothing covering the subject's body. The transducer may include suitable materials for attaching the transducer to the subject's body. For example, the suitable materials may include cloth, foam, flexible plastic, and/or a conductive medical gel. The transducer may be conductive or non-conductive.

The transducer may include any desired number of electrode elements. Various shapes, sizes, and materials may be used for the electrode elements. Any constructions for implementing the transducer (or electric field generating device) for use with embodiments of the invention may be used as long as they are capable of (a) delivering TTFields to the subject's body and (b) being positioned at the locations specified herein. In some embodiments, at least one electrode element of the first, the second, the third, or the fourth transducer may include at least one ceramic disk that is adapted to generate an alternating electric field. In some embodiments, at least one electrode element of the first, the second, the third, or the fourth transducer may include a polymer film that is adapted to generate an alternating electric field.

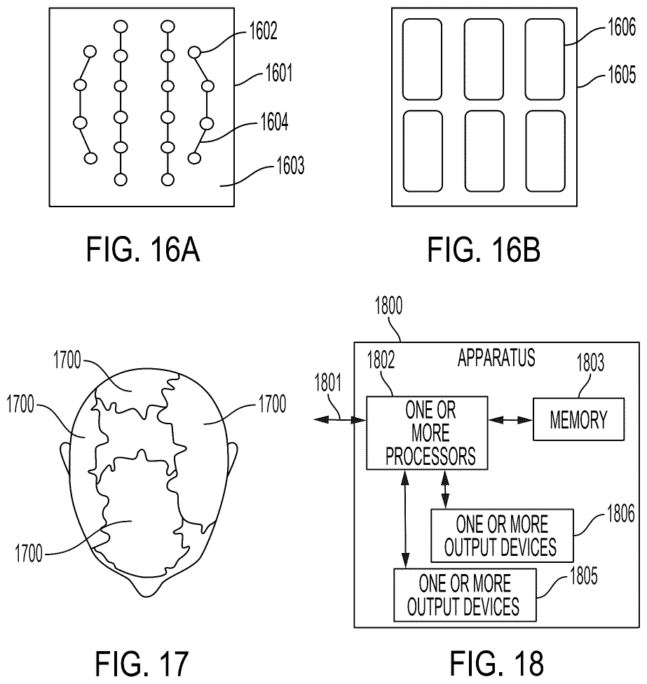

FIG. 16A illustrates a schematic view of an exemplary design of a transducer for applying alternating electric fields. Transducer 1601 includes twenty electrode elements 1602, which are positioned on substrate 1603, and electrode elements 1602 are electrically and physically connected to one another through a conductive wiring 1604. In some embodiments, electrode elements 1602 may include a ceramic disk.

FIG. 16B illustrates a schematic view of an exemplary design of a transducer for applying alternating electric fields. Transducer 1605 may include substantially flat electrode elements 1606. In some embodiments, electrode elements 1606 are non-ceramic dielectric materials positioned over flat conductors. Examples of non-ceramic dielectric materials positioned over flat conductors may include polymer films disposed over pads on a printed circuit board or over substantially planar pieces of metal. In some embodiments, such polymer films have a high dielectric constant, such as, for example, a dielectric constant greater than 10. In some embodiments, electrode elements 1606 may have various shapes. For example, the electrode elements may be triangular, rectangular, circular, oval, ovaloid, ovoid, or elliptical in shape or substantially triangular, substantially rectangular, substantially circular, substantially oval, substantially ovaloid, substantially ovoid, or substantially elliptical in shape. In some embodiments, each of electrode elements 1606 may have a same shape, similar shapes, and/or different shapes.

FIG. 18 depicts an example computer apparatus 1800 for use with one or more embodiments described herein. As an example, apparatus 1800 may be a computer to implement certain inventive techniques disclosed herein, such as determining transducer locations for delivery of TTFields. As an example, method 100 of FIG. 1A, the method 120 of FIG. 1B, the method 600 of FIG. 6, and/or the method 1000 of FIG. 10 may be performed by a computer, such as the apparatus 1800. As an example, the method 100 of FIG. 1A, the method 120 of FIG. 1B, the method 600 of FIG. 6, the steps of FIG. 7, the steps of FIG. 8 and/or the method 1000 of FIG. 10 may be performed by a computer, such as apparatus 1800. In some embodiments, controller 1510 of FIG. 15 may be implemented with apparatus 1800. As an example, some or all of the steps in the method illustrated in FIGS. 1A, 1B, 6-8 and/or 10 may be performed on a single apparatus 1800. As an example, the steps in the method illustrated in FIGS. 1A, 1B, 6-8 and/or 10 may be performed by one, two, three, four, or more apparatuses 1800. As an example, the apparatus 1800 may be a portable computer device (e.g., a mobile device, a tablet or a laptop).

The apparatus 1800 may include one or more processors 1802, memory 1803, one or more input devices 1805, and one or more output devices 1806.

Input to the apparatus 1800 may be provided by one or more input devices 1805, provided from one or more input devices in communication with the apparatus 1800 via link 1801 (e.g., a wired link or a wireless link; e.g., with a direct connection or over a network), and/or provided from another computer(s) in communication with the apparatus 1800 via link 1801.

Output for the apparatus 1800 may be provided by one or more output devices 1806, provided to one or more output devices in communication with the apparatus 1800 via link 1801, and/or provided from another computer(s) in communication with the apparatus 1800 via link 1801. The one or more output devices 1806 may provide the status of the operation according to some embodiments described herein, such as transducer array selection, voltages being generated, and other operational information. The output device(s) 1806 may provide visualization data according to some embodiments described herein. The one or more output devices 1806 may include one more displays and one or more speakers. In some embodiments, the output device(s) 1806 may display healthy models, a model of the abnormality, recommended locations, predetermined locations and/or associated information as described with respect to FIGS. 1A, 1B, and 6-11, and/or another suitable outputs associated with determining transducer locations for delivery of tumor treating fields according to one or more embodiments described herein.

In some embodiments, one or more input devices 1805 and one or more output devices 1806 may be combined into one or more unitary input/output devices (e.g., a touch screen).

In some embodiments, based on input from one or more input devices 1805 or input from outside the apparatus 1800 via the link 1801, the one or more processors 1802 may perform operations as described herein. For example, based on input 1801, the one or more processors 1802 may determine transducer locations for delivery of tumor treating fields to implement one or more embodiments described herein. For example, based on input 1801, the one or more processors 1802 may generate control signals to control the voltage generator to implement one or more embodiments described herein. As an example, user input may be received from the one or more input devices 1805. As an example, input may be from another computer in communication with the apparatus 1800 via link 1801. As an example, input may be from one or more input devices in communication with the apparatus 1800 via link 1801.

In some embodiments, the one or more processors 1802 may perform operations as described herein and provide results of the operations as output. As an example, output may be provided to the one or more output devices 1806. As an example, output may be provided to another computer in communication with the apparatus 1800 via link 1801. As an example, output may be provided to one or more output devices in communication with the apparatus 1800 via link 1801.

The memory 1803 may be accessible by the one or more processors 1802 so that the one or more processors 1802 may read information from and write information to the memory 1803. The memory 1803 may store instructions that, when executed by the one or more processors 1802, implement one or more embodiments described herein. The memory 1803 may be a non-transitory computer readable medium (or a non-transitory processor readable medium) containing a set of instructions thereon for determining transducer locations for delivery of tumor treating fields, wherein when executed by a processor (such as one or more processors 1802), the instructions cause the processor to perform one or more methods discussed herein. As an example, the apparatus 1800 may be a computer, and memory of the computer may store an app, a program, or a software to perform embodiments described herein.

The apparatus 1800 may be an apparatus for determining transducer locations for delivery of tumor treating fields, the apparatus including: one or more processors (such as one or more processors 1802); and memory (such as memory 1803) accessible by the one or more processors, the memory storing instructions that when executed by the one or more processors, cause the apparatus to perform one or more methods described herein.

The memory 1803 may be a non-transitory processor readable medium containing a set of instructions thereon for determining transducer locations for delivery of tumor treating fields, wherein when executed by one or more processors (such as one or more processors 1802), the instructions cause the one or more processors to perform one or more methods described herein.

### ILLUSTRATIVE EMBODIMENTS

The invention includes other illustrative embodiments (“Embodiments”) as follows.

Embodiment 1: A computer-implemented method for determining transducer locations for delivery of tumor treating fields based on models of healthy subjects, the method comprising: receiving a plurality of external measurements of the subject, the plurality of external measurements not derived from a medical image of the subject; receiving a selection of a general location of an abnormality of the subject, the general location selected from a plurality of general locations of the subject; selecting a healthy model from a plurality of healthy models, the selected healthy model being representative of the subject, the selection based on the external measurements of the subject; selecting locations on the selected healthy model to place transducers to treat the abnormality of the subject using tumor treating fields, the selection based on the selected general location of the abnormality of the subject; and providing the selected locations to place transducers to treat the abnormality of the subject using tumor treating fields.

Embodiment 2: The method of Embodiment 1, wherein the external measurements of the subject are from a physical measurement of the subject.

Embodiment 3: The method of Embodiment 1, wherein the external measurements of the subject include: a weight of the subject; a height of the subject; a chest circumference of the subject; a waist circumference of the subject; a bust circumference of the subject; a shoulder span of the subject; a measurement from C7 to iliac-crests mid; a measurement from the clavicle to a nipple of the subject; and a measurement from the jugular notch to the xiphoid process

Embodiment 3A: The method of Embodiment 1, wherein the external measurements of the subject include at least five of: a weight of the subject; a height of the subject; a chest circumference of the subject; a waist circumference of the subject; a bust circumference of the subject; a shoulder span of the subject; a measurement from C7 to iliac-crests mid; a measurement from the clavicle to a nipple of the subject; and a measurement from the jugular notch to the xiphoid process.

Embodiment 4: The method of Embodiment 1, wherein the external measurements of the subject are received from a user interface for a user to enter the external measurements of the subject.

Embodiment 5: The method of Embodiment 1, wherein the external measurements of the subject are calculated using an app on a personal computing device, wherein the app calculates the external measurements of the subject based on images of the subject obtained with a camera on the personal computing device.

Embodiment 5A: The method of Embodiment 5, wherein the external measurements of the subject are received from the app on the personal computing device.

Embodiment 6: The method of Embodiment 1, wherein the plurality of general locations are in a torso, a thorax, or an abdomen of the subject.

Embodiment 7: The method of Embodiment 1, wherein the plurality of general locations comprise clinically significant regions of an organ of the subject.

Embodiment 7A: The method of Embodiment 1, wherein the plurality of general locations comprise a superior lobe in a left lung, an inferior lobe in the left lung, a superior lobe in a right lung, a middle lobe in the right lung, and an inferior lobe in the right lung.

Embodiment 8: The method of Embodiment 1, wherein the plurality of general locations comprise areas divided by a grid of a region of the subject where the abnormality of the subject is located.

Embodiment 8A: The method of Embodiment 1, wherein the plurality of general locations comprise an upper area, a middle area, and a lower area in a left lung and an upper area, a middle area, and a lower area in a right lung.

Embodiment 8B: The method of Embodiment 1, wherein the plurality of general locations comprise a grid of six areas in a left lung and a grid of six areas in a right lung.

Embodiment 8C: The method of Embodiment 1, wherein the plurality of general locations comprise a grid of nine areas in an abdomen of the subject.

Embodiment 8D: The method of Embodiment 1, wherein the general location of the abnormality of the subject is received from a user interface providing at least one of a list or an image of the plurality of general locations of the subject, wherein the general location is selectable by a user from at least one of the list or the image.

Embodiment 9: The method of Embodiment 1, wherein selecting the healthy model comprises: comparing the external measurements of the subject to external measurements for each of the plurality of healthy models; and selecting the healthy model being representative of the subject as the healthy model having external measurements most similar to the external measurements of the subject.

Embodiment 9A: The method of Embodiment 1, wherein each of the plurality of healthy models is representative of a group of healthy subjects without abnormalities.

Embodiment 10: The method of Embodiment 1, wherein the plurality of healthy models is based on models of healthy subjects clustered into groups according to external measurements of the healthy subjects.

Embodiment 10A: The method of Embodiment 1, wherein each of the healthy models is defined by a vector having a number of variables corresponding to external measurements for the healthy model.

Embodiment 11: The method of Embodiment 1, further comprising, prior to receiving the external measurements of the subject, generating the plurality of healthy models.

Embodiment 12: The method of Embodiment 11, wherein generating the plurality of healthy models comprises: receiving training data for a plurality of healthy subjects, wherein the training data comprises external measurements for each of the plurality of healthy subjects; analyzing the training data to identify commonalities among the healthy subjects; clustering the healthy subjects into clusters based at least in part on the commonalities among the healthy subjects; and selecting, for each cluster, one of the plurality of healthy models based at least in part on the training data for the healthy subjects that are within the cluster, wherein the plurality of healthy models comprises the selected healthy model for each cluster.

Embodiment 13: The method of Embodiment 12, wherein selecting, for each cluster, one of the plurality of healthy models comprises: determining a center in n-dimensional space for each cluster, wherein each cluster is defined by an n-dimensional vector, wherein the dimensions of the n-dimensional vector correspond to the external measurements; selecting the model of the healthy subjects in the cluster closest to the center of the cluster as the healthy model for the cluster.

Embodiment 13A: The method of Embodiment 12, wherein selecting, for each cluster, one of the plurality of healthy models comprises generating, for each cluster, the healthy model using information regarding at least two of the healthy subjects within the cluster.

Embodiment 14: The method of Embodiment 12, wherein each of the healthy subjects has at least one medical image associated therewith, wherein external measurements for the healthy subjects are not derived from the medical images associated with the healthy subjects.

Embodiment 15: The method of Embodiment 12, wherein analyzing the training data comprises performing a principal component analysis to identify the commonalities among the plurality of healthy subjects.

Embodiment 15A: The method of Embodiment 12, wherein the commonalities are based on principal components defined for each of the healthy subjects.

Embodiment 15B: The method of Embodiment 12, wherein the principal components are defined by a subject matter expert.

Embodiment 15C: The method of Embodiment 12, wherein the principal components are defined automatically based on analyzing the training data.

Embodiment 15D: The method of Embodiment 12, wherein the clustering is performed using k-means clustering.

Embodiment 16: The method of Embodiment 1, further comprising, prior to receiving the external measurements of the subject, predetermining locations on the healthy models to place transducers to deliver tumor treating fields to each of the plurality of general locations for each healthy model, wherein the selected locations to place transducers to treat the abnormality of the subject using tumor treating fields are the corresponding predetermined locations on the selected healthy model for the selected general location.

Embodiment 17: The method of Embodiment 16, wherein predetermining locations on the healthy models to place transducers is based at least in part on conductivities for at least one tissue type included in the healthy models.

Embodiment 17A: The method of Embodiment 16, wherein the healthy models define healthy tissue having electrical properties, wherein predetermining locations on the healthy models to place transducers is based on at least in part on the electrical properties.

Embodiment 18: The method of Embodiment 16, wherein predetermining locations on the healthy models to place transducers comprises: receiving a plurality of transducer layouts for the healthy models, wherein each transducer layout comprises locations on the healthy model to place transducers to deliver tumor treating fields to the healthy model; performing electric field simulations for the healthy models for the plurality of transducer layouts, wherein the healthy models comprise medical images segmented into tissue types, wherein each tissue type is assigned an electrical conductivity, wherein the healthy models are not modified to include abnormal tissue; receiving a plurality of regions of interest for the healthy models, wherein each of the regions of interest for the healthy models corresponds to one of the general locations of the subject; and for each of the plurality of regions of interest for the healthy models, calculating, for each of the plurality of transducer layouts, a dosage of tumor treating fields treatment in the region of interest of the healthy model based on the performed electric field simulations; and selecting the transducer layout as the transducer layout for the region of interest based on the calculated dosages of tumor treating fields treatment for the plurality of transducer layouts.

Embodiment 18A: The method of Embodiment 18, wherein calculating the dosage of tumor treating fields treatment in the region of interest of the healthy model comprises averaging electric field strength for voxels of the medical image in the region of interest.

Embodiment 18B: The method of Embodiment 18, wherein the medical image is at least one of a computed tomography (CT) image, a magnetic resonance imaging (MRI) medical image, or a positron emission tomography (PET) medical image.

Embodiment 18C: The method of Embodiment 1, wherein providing the selected locations to place transducers comprises at least one of: presenting an image of the subject with identifications of the selected locations to place transducers; presenting an image of a generic subject with identifications of the selected locations to place transducers; or presenting a description of the selected locations to place transducers.

Embodiment 18D: The method of Embodiment 1, wherein the abnormality is a tumor.

Embodiment 18E: An apparatus for determining transducer locations for delivery of tumor treating fields based on models of healthy subjects, the apparatus comprising: one or more processors; and a memory accessible by the one or more processors, the memory storing instructions that when executed by the one or more processors, cause the apparatus to: receiving a plurality of external measurements of the subject, the plurality of external measurements not derived from a medical image of the subject; receiving a selection of a general location of an abnormality of the subject, the general location selected from a plurality of general locations of the subject; selecting a healthy model from a plurality of healthy models, the selected healthy model being representative of the subject, the selection based on the external measurements of the subject; selecting locations on the selected healthy model to place transducers to treat the abnormality of the subject using tumor treating fields, the selection based on the selected general location of the abnormality of the subject; and providing the selected locations to place transducers to treat the abnormality of the subject using tumor treating fields.

Embodiment 19: A non-transitory processor readable medium containing a set of instructions thereon for determining transducer locations for delivery of tumor treating fields based on models of healthy subjects, wherein when executed by a processor, the instructions cause the processor to: receiving a plurality of external measurements of the subject, the plurality of external measurements not derived from a medical image of the subject; receiving a selection of a general location of an abnormality of the subject, the general location selected from a plurality of general locations of the subject; selecting a healthy model from a plurality of healthy models, the selected healthy model being representative of the subject, the selection based on the external measurements of the subject; selecting locations on the selected healthy model to place transducers to treat the abnormality of the subject using tumor treating fields, the selection based on the selected general location of the abnormality of the subject; and providing the selected locations to place transducers to treat the abnormality of the subject using tumor treating fields.

Embodiment 20: An apparatus for determining transducer locations for delivery of tumor treating fields based on models of healthy subjects, the apparatus comprising: one or more processors; and a memory accessible by the one or more processors, the memory storing a database of a plurality of healthy models, wherein the healthy models are indexed by external measurements, wherein external measurements differentiate the healthy models from each other, wherein the external measurements of each healthy model represent a corresponding representative group of healthy subjects, wherein each of the plurality of healthy models are indexed by general locations for each healthy model, wherein each general location for each healthy model identifies predetermined locations on the healthy models to place transducers to deliver tumor treating fields to the general location, the memory further storing instructions that when executed by the one or more processors, cause the apparatus to: select a healthy model from the plurality of healthy models, the selected healthy model being representative of a subject, the selection based on external measurements of the subject, the external measurements of the subject not derived from a medical image of the subject; and select locations on the selected healthy model to place transducers to treat an abnormality of the subject using tumor treating fields, the selection based on a general location of the abnormality of the subject. the selection locations selected from the predetermined locations of the selected healthy model; and provide the selected locations to place transducers to treat the abnormality of the subject using tumor treating fields.

Embodiment 20A: The apparatus of Embodiment 20, wherein the predetermined locations on the healthy models to place transducers to deliver tumor treating fields are based on: electric field simulations of segmented medical images of the healthy models for the predetermined locations, and calculated dosages of tumor treating fields treatment in the general locations of the healthy model based on the performed electric field simulations.

Embodiment 21: A method, machine, manufacture, and/or system substantially as shown and described.

Embodiment 101: A computer-implemented method for determining transducer locations for delivery of tumor treating fields based on models of healthy subjects, the method comprising: receiving category information of the subject, wherein the category information of the subject includes at least two qualitative properties of the subject, wherein the category information of the subject does not include quantitative properties of the subject; receiving a selection of a general location of an abnormality of the subject, the general location selected from a plurality of general locations of the subject; selecting a healthy model from a plurality of healthy models, the selected healthy model being representative of the subject, the selection based on the category information of the subject; selecting locations on the selected healthy model to place transducers to treat the abnormality of the subject using tumor treating fields, the selection based on the selected general location of the abnormality of the subject; and providing the selected locations to place transducers to treat the abnormality of the subject using tumor treating fields.

Embodiment 102: The method of Embodiment 101, wherein the category information does not include external measurements of the subject, wherein the category information does not include internal measurements of the subject, wherein the category information does not include information derived from a medical image of the subject.

Embodiment 102A: The method of Embodiment 101, wherein the category information comprises gender of the subject and size of the subject.

Embodiment 103: The method of Embodiment 101, wherein the category information consists of gender of the subject and size of the subject.

Embodiment 103A: The method of Embodiment 103, wherein the category information for the size of the subject is selected from three options, wherein the three options are small, medium, and large.

Embodiment 103B: The method of Embodiment 103, wherein the category information for the size of the subject includes a height of the subject and a girth of the subject.

Embodiment 103C: The method of Embodiment 103B, wherein the category information for the height of the subject is selected from three options, wherein the three options are short, medium, and tall, and wherein the category information for the girth of the subject is selected from three options, wherein the three options are thin, average, and heavy.

Embodiment 104: The method of Embodiment 101, wherein the category information of the subject is received from a user interface for a user to enter the category information of the subject.

Embodiment 104A: The method of Embodiment 104, wherein the user interface includes a user-selectable option for gender of the subject, wherein the user interface includes a user-selectable option for a size of the subject.

Embodiment 105: The method of Embodiment 101, further comprising: receiving one or more external measurements of the subject; and converting the one or more external measurements of the subject to the category information of the subject.

Embodiment 105A: The method of Embodiment 105, wherein the one or more external measurements of the subject comprises at least one of a height of the subject, a weight of the subject, or a body mass index of the subject, and wherein the category information of the subject comprises size of the subject.

Embodiment 105B: The method of Embodiment 105, wherein the external measurements of the subject are received from a user interface for a user to enter the one or more external measurements of the subject.

Embodiment 105C: The method of Embodiment 105, wherein the one or more external measurements of the subject are calculated using an app on a personal computing device, wherein the app calculates the one or more external measurements of the subject based on images of the subject obtained with a camera on the personal computing device.

Embodiment 105D: The method of Embodiment 105C, wherein the one or more external measurements of the subject are received from the app on the personal computing device.

Embodiment 106: The method of Embodiment 101, wherein the plurality of general locations are in a torso, a thorax, or an abdomen of the subject.

Embodiment 107: The method of Embodiment 101, wherein the plurality of general locations comprise clinically significant well known regions of an organ of the subject.

Embodiment 107A: The method of Embodiment 101, wherein the plurality of general locations comprise a superior lobe in a left lung, an inferior lobe in the left lung, a superior lobe in a right lung, a middle lob in the right lung, and an inferior lobe in the right lung.

Embodiment 108: The method of Embodiment 101, wherein the plurality of general locations comprise areas divided by a grid of a region of the subject where the abnormality of the subject is located.

Embodiment 108A: The method of Embodiment 101, wherein the plurality of general locations comprise an upper area, a middle area, and a lower area in a left lung and an upper area, a middle area, and a lower area in a right lung.

Embodiment 108B: The method of Embodiment 101, wherein the plurality of general locations comprise a grid of six areas in a left lung and a grid of six areas in a right lung.

Embodiment 108C: The method of Embodiment 101, wherein the plurality of general locations comprise a grid of nine areas in an abdomen of the subject.

Embodiment 108D: The method of Embodiment 101, wherein the general location of the abnormality of the subject is received from a user interface providing a list of the plurality of general locations of the subject, wherein the general location is selectable by a user from the list.

Embodiment 109: The method of Embodiment 101, wherein selecting the healthy model is based on applying a selection tree to the category information of the subject.

Embodiment 109A: The method of Embodiment 101, wherein selecting the healthy model comprises: comparing the category information of the subject to category information for each of the plurality of healthy models; and selecting the healthy model being representative of the subject as the healthy model having category information most similar to the category information of the subject.

Embodiment 109B: The method of Embodiment 101, wherein each of the plurality of healthy models is representative of a group of healthy subjects without abnormalities.

Embodiment 110: The method of Embodiment 101, wherein the plurality of healthy models is based on models of healthy subjects clustered into groups according to category information of the healthy subjects.

Embodiment 110A: The method of Embodiment 101, wherein each of the healthy models is defined by a vector having a number of variables corresponding to category information for the healthy model.

Embodiment 111: The method of Embodiment 101, further comprising, prior to receiving the category information of the subject, generating the plurality of healthy models.

Embodiment 112: The method of Embodiment 111, wherein generating the plurality of healthy models comprises: receiving training data for a plurality of healthy subjects, wherein the training data comprises category information for each of the plurality of healthy subjects; analyzing the training data to identify commonalities among the healthy subjects; clustering the healthy subjects into clusters based at least in part on the commonalities among the healthy subjects; and selecting, for each cluster, one of the plurality of healthy models based at least in part on the training data for the healthy subjects that are within the cluster, wherein the plurality of healthy models comprises the selected healthy model for each cluster.

Embodiment 113: The method of Embodiment 112, wherein selecting, for each cluster, one of the plurality of healthy models comprises: determining a center in n-dimensional space for each cluster, wherein each cluster is defined by an n-dimensional vector, wherein the dimensions of the n-dimensional vector correspond to the category information; selecting the model of the healthy subjects in the cluster closest to the center of the cluster as the healthy model for the cluster.

Embodiment 113A: The method of Embodiment 112, wherein selecting, for each cluster, one of the plurality of healthy models comprises generating, for each cluster, the healthy model using information regarding at least two of the healthy subjects within the cluster.

Embodiment 114: The method of Embodiment 112, wherein each of the healthy subjects has at least one medical image associated therewith, wherein category information for the healthy subjects is not derived from the medical images associated with the healthy subjects.

Embodiment 115: The method of Embodiment 112, wherein analyzing the training data comprises performing a principal component analysis to identify the commonalities among the plurality of healthy subjects.

Embodiment 115A: The method of Embodiment 112, wherein the commonalities are based on principal components defined for each of the healthy subjects.

Embodiment 115B: The method of Embodiment 112, wherein the principal components are defined by a subject matter expert.

Embodiment 115C: The method of Embodiment 112, wherein the principal components are defined automatically based on analyzing the training data.

Embodiment 115D: The method of Embodiment 112, wherein the clustering is performed using k-means clustering.

Embodiment 116: The method of Embodiment 101, further comprising, prior to receiving the category information of the subject, predetermining locations on the healthy models to place transducers to deliver tumor treating fields to each of the plurality of general locations for each healthy model, wherein the selected locations to place transducers to treat the abnormality of the subject using tumor treating fields are the corresponding predetermined locations on the selected healthy model for the selected general location.

Embodiment 117: The method of Embodiment 116, wherein predetermining locations on the healthy models to place transducers is based at least in part on conductivities for at least one tissue type included in the healthy models.

Embodiment 117A: The method of Embodiment 116, wherein the healthy models define healthy tissue having electrical properties, wherein predetermining locations on the healthy models to place transducers is based on at least in part on the electrical properties.

Embodiment 118: The method of Embodiment 116, wherein predetermining locations on the healthy models to place transducers comprises: receiving a plurality of transducer layouts for the healthy models, wherein each transducer layout comprises locations on the healthy model to place transducers to deliver tumor treating fields to the healthy model; performing electric field simulations for the healthy models for the plurality of transducer layouts, wherein the healthy models comprise medical images segmented into tissue types, wherein each tissue type is assigned an electrical conductivity, wherein the healthy models are not modified to include abnormal tissue; receiving a plurality of regions of interest for the healthy models, wherein each of the regions of interest for the healthy models corresponds to one of the general locations of the subject; and for each of the plurality of regions of interest for the healthy models, calculating, for each of the plurality of transducer layouts, a dosage of tumor treating fields treatment in the region of interest of the healthy model based on the performed electric field simulations; and selecting the transducer layout as the transducer layout for the region of interest based on the calculated dosages of tumor treating fields treatment for the plurality of transducer layouts.

Embodiment 118A: The method of Embodiment 118, wherein calculating the dosage of tumor treating fields treatment in the region of interest of the healthy model comprises averaging electric field strength for voxels of the medical image in the region of interest.

Embodiment 118B: The method of Embodiment 118, wherein the medical image is at least one of a computed tomography (CT) image, a magnetic resonance imaging (MRI) medical image, or a positron emission tomography (PET) medical image.

Embodiment 118C: The method of Embodiment 101, wherein providing the selected locations to place transducers comprises at least one of: presenting an image of the subject with identifications of the selected locations to place transducers; presenting an image of a generic subject with identifications of the selected locations to place transducers; or presenting a description of the selected locations to place transducers.

Embodiment 118D: The method of Embodiment 101, wherein the abnormality is a tumor.

Embodiment 118E: An apparatus for determining transducer locations for delivery of tumor treating fields based on models of healthy subjects, the apparatus comprising: one or more processors; and a memory accessible by the one or more processors, the memory storing instructions that when executed by the one or more processors, cause the apparatus to: receiving category information of the subject, wherein the category information of the subject includes at least two qualitative properties of the subject, wherein the category information of the subject does not include quantitative properties of the subject; receiving a selection of a general location of an abnormality of the subject, the general location selected from a plurality of general locations of the subject; selecting a healthy model from a plurality of healthy models, the selected healthy model being representative of the subject, the selection based on the category information of the subject; selecting locations on the selected healthy model to place transducers to treat the abnormality of the subject using tumor treating fields, the selection based on the selected general location of the abnormality of the subject; and providing the selected locations to place transducers to treat the abnormality of the subject using tumor treating fields.

Embodiment 119: A non-transitory processor readable medium containing a set of instructions thereon for determining transducer locations for delivery of tumor treating fields based on models of healthy subjects, wherein when executed by a processor, the instructions cause the processor to: receiving category information of the subject, wherein the category information of the subject includes at least two qualitative properties of the subject, wherein the category information of the subject does not include quantitative properties of the subject; receiving a selection of a general location of an abnormality of the subject, the general location selected from a plurality of general locations of the subject; selecting a healthy model from a plurality of healthy models, the selected healthy model being representative of the subject, the selection based on the category information of the subject; selecting locations on the selected healthy model to place transducers to treat the abnormality of the subject using tumor treating fields, the selection based on the selected general location of the abnormality of the subject; and providing the selected locations to place transducers to treat the abnormality of the subject using tumor treating fields.

Embodiment 120: An apparatus for determining transducer locations for delivery of tumor treating fields based on models of healthy subjects, the apparatus comprising: one or more processors; and a memory accessible by the one or more processors, the memory storing a database of a plurality of healthy models, wherein the healthy models are indexed by category information, wherein category information differentiates the healthy models from each other, wherein the category information of each healthy model represents a corresponding representative group of healthy subjects, wherein each of the plurality of healthy models are indexed by general locations for each healthy model, wherein each general location for each healthy model identifies predetermined locations on the healthy models to place transducers to deliver tumor treating fields to the general location, the memory further storing instructions that when executed by the one or more processors, cause the apparatus to: select a healthy model from the plurality of healthy models, the selected healthy model being representative of a subject, the selection based on selected category information of the subject; and select locations on the selected healthy model to place transducers to treat an abnormality of the subject using tumor treating fields, the selection based on a general location of the abnormality of the subject. the selection locations selected from the predetermined locations of the selected healthy model; and provide the selected locations to place transducers to treat the abnormality of the subject using tumor treating fields.

Embodiment 120A: The apparatus of Embodiment 120, wherein the predetermined locations on the healthy models to place transducers to deliver tumor treating fields are based on: electric field simulations of segmented medical images of the healthy models for the predetermined locations, and calculated dosages of tumor treating fields treatment in the general locations of the healthy model based on the performed electric field simulations.

Embodiment 121: A method, machine, manufacture, and/or system substantially as shown and described.

Optionally, for each embodiment described herein, the voltage generation components supply the transducers with an electrical signal having an alternating current waveform at frequencies in a range from about 50 kHz to about 1 MHz and appropriate to deliver TTFields treatment to the subject's body.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention need not be limited to the described embodiments, but that it has the full scope defined by the language of the following claims, and equivalents thereof.

What is claimed is:

1. A computer-implemented method for determining transducer locations for delivery of tumor treating fields based on models of healthy subjects, the method comprising:
receiving a plurality of external measurements of the subject, the plurality of external measurements not derived from a medical image of the subject;
receiving a selection of a general location of an abnormality of the subject, the general location selected from a plurality of general locations of the subject;
selecting a healthy model from a plurality of healthy models, the selected healthy model being representative of the subject, the selection based on the external measurements of the subject;
selecting locations on the selected healthy model to place transducers to treat the abnormality of the subject using tumor treating fields, the selection based on the selected general location of the abnormality of the subject; and
providing the selected locations to place transducers to treat the abnormality of the subject using tumor treating fields.

2. The method of claim 1, wherein the external measurements of the subject are from a physical measurement of the subject.

3. The method of claim 1, wherein the external measurements of the subject include:
a weight of the subject; a height of the subject; a chest circumference of the subject; a waist circumference of the subject; a bust circumference of the subject; a shoulder span of the subject;
a measurement from C7 to iliac-crests mid; a measurement from the clavicle to a nipple of the subject; and a measurement from the jugular notch to the xiphoid process.

4. The method of claim 1, wherein the external measurements of the subject are received from a user interface for a user to enter the external measurements of the subject.

5. The method of claim 1, wherein the external measurements of the subject are calculated using an app on a personal computing device, wherein the app calculates the external measurements of the subject based on images of the subject obtained with a camera on the personal computing device.

6. The method of claim 1, wherein the plurality of general locations are in a torso, a thorax, or an abdomen of the subject.

7. The method of claim 1, wherein the plurality of general locations comprise clinically significant regions of an organ of the subject.

8. The method of claim 1, wherein the plurality of general locations comprise areas divided by a grid of a region of the subject where the abnormality of the subject is located.

9. The method of claim 1, wherein selecting the healthy model comprises:
comparing the external measurements of the subject to external measurements for each of the plurality of healthy models; and
selecting the healthy model being representative of the subject as the healthy model having external measurements most similar to the external measurements of the subject.

10. The method of claim 1, wherein the plurality of healthy models is based on models of healthy subjects clustered into groups according to external measurements of the healthy subjects.

11. The method of claim 1, further comprising, prior to receiving the external measurements of the subject, generating the plurality of healthy models.

12. The method of claim 11, wherein generating the plurality of healthy models comprises:
receiving training data for a plurality of healthy subjects, wherein the training data comprises external measurements for each of the plurality of healthy subjects;
analyzing the training data to identify commonalities among the healthy subjects;
clustering the healthy subjects into clusters based at least in part on the commonalities among the healthy subjects; and
selecting, for each cluster, one of the plurality of healthy models based at least in part on the training data for the healthy subjects that are within the cluster, wherein the plurality of healthy models comprises the selected healthy model for each cluster.

13. The method of claim 12, wherein selecting, for each cluster, one of the plurality of healthy models comprises:
determining a center in n-dimensional space for each cluster, wherein each cluster is defined by an n-dimensional vector, wherein the dimensions of the n-dimensional vector correspond to the external measurements;
selecting the model of the healthy subjects in the cluster closest to the center of the cluster as the healthy model for the cluster.

14. The method of claim 12, wherein each of the healthy subjects has at least one medical image associated therewith, wherein external measurements for the healthy subjects are not derived from the medical images associated with the healthy subjects.

15. The method of claim 12, wherein analyzing the training data comprises performing a principal component analysis to identify the commonalities among the plurality of healthy subjects.

16. The method of claim 1, further comprising, prior to receiving the external measurements of the subject, predetermining locations on the healthy models to place transducers to deliver tumor treating fields to each of the plurality of general locations for each healthy model,
wherein the selected locations to place transducers to treat the abnormality of the subject using tumor treating fields are the corresponding predetermined locations on the selected healthy model for the selected general location.

17. The method of claim 16, wherein predetermining locations on the healthy models to place transducers is based at least in part on conductivities for at least one tissue type included in the healthy models.

18. The method of claim 16, wherein predetermining locations on the healthy models to place transducers comprises:
receiving a plurality of transducer layouts for the healthy models, wherein each transducer layout comprises locations on the healthy model to place transducers to deliver tumor treating fields to the healthy model;
performing electric field simulations for the healthy models for the plurality of transducer layouts, wherein the healthy models comprise medical images segmented into tissue types, wherein each tissue type is assigned an electrical conductivity, wherein the healthy models are not modified to include abnormal tissue;
receiving a plurality of regions of interest for the healthy models, wherein each of the regions of interest for the healthy models corresponds to one of the general locations of the subject; and
for each of the plurality of regions of interest for the healthy models,
calculating, for each of the plurality of transducer layouts, a dosage of tumor treating fields treatment in the region of interest of the healthy model based on the performed electric field simulations; and
selecting the transducer layout as the transducer layout for the region of interest based on the calculated dosages of tumor treating fields treatment for the plurality of transducer layouts.

19. A non-transitory processor readable medium containing a set of instructions thereon for determining transducer locations for delivery of tumor treating fields based on models of healthy subjects, wherein when executed by a processor, the instructions cause the processor to:
receiving a plurality of external measurements of the subject, the plurality of external measurements not derived from a medical image of the subject;
receiving a selection of a general location of an abnormality of the subject, the general location selected from a plurality of general locations of the subject;
selecting a healthy model from a plurality of healthy models, the selected healthy model being representative of the subject, the selection based on the external measurements of the subject;
selecting locations on the selected healthy model to place transducers to treat the abnormality of the subject using tumor treating fields, the selection based on the selected general location of the abnormality of the subject; and
providing the selected locations to place transducers to treat the abnormality of the subject using tumor treating fields.

20. An apparatus for determining transducer locations for delivery of tumor treating fields based on models of healthy subjects, the apparatus comprising:
one or more processors; and
a memory accessible by the one or more processors, the memory storing a database of a plurality of healthy models,
wherein the healthy models are indexed by external measurements, wherein external measurements differentiate the healthy models from each other, wherein the external measurements of each healthy model represent a corresponding representative group of healthy subjects,
wherein each of the plurality of healthy models are indexed by general locations for each healthy model, wherein each general location for each healthy model identifies predetermined locations on the healthy models to place transducers to deliver tumor treating fields to the general location,
the memory further storing instructions that when executed by the one or more processors, cause the apparatus to:
select a healthy model from the plurality of healthy models, the selected healthy model being representative of a subject, the selection based on external measurements of the subject, the external measurements of the subject not derived from a medical image of the subject; and
select locations on the selected healthy model to place transducers to treat an abnormality of the subject using tumor treating fields, the selection based on a general location of the abnormality of the subject, the selection locations selected from the predetermined locations of the selected healthy model; and
provide the selected locations to place transducers to treat the abnormality of the subject using tumor treating fields.
